(19) United States
(12) Patent Application Publication
(10) Pub. No.: US 2003/0175250 A1
(43) Pub. Date: Sep. 18, 2003

(54) Isolated peptides which bind to HLA molecules and uses thereof

(76) Inventors: Elke Jager, Frankfurt am Main (DE); Alexander Knuth, Frankfurt am Main (DE); Lloyd J. Old, New York, NY (US); Sacha Gnjatic, New York, NY (US)

Correspondence Address: Mary Anne Schofield, FULBRIGHT & JAWORSKI L.L.P., 801 Pennsylvania Avenue, N.W., Washington, DC, 20004, US

(21) Appl. No.: 10/364,614

(22) Filed: Feb. 12, 2003

Related U.S. Application Data
(60) Provisional application No. 60/355,828, filed on Feb. 13, 2002.

(51) Int. Cl. G01N 33/53; G01N 33/567; C07H 21/04; A61K 45/00; C12P 21/02; C12N 5/06; C07K 7/08

(52) U.S. Cl. 424/93.7; 435/7.2; 435/69.1; 435/320.1; 435/325; 530/327; 530/328; 530/350; 536/23.5

(57) ABSTRACT

This invention relates to isolated peptides that bind to an HLA molecule particularly HLA-A3, HLA-B35 and/or HLA-B51 and stimulate cytolytic T cells specific for complexes of the peptide and the HLA molecule. This invention also relates to CTLs, antibodies, antibody fragments and T cell receptors that are specific for HLA/peptide complexes, and to methods of using the peptides, CTLs, antibodies and receptors.

This application claims priority to U.S. provisional application No. 60/355,828 filed Feb. 13, 2002 and incorporated herein by reference in its entirety.

### FIELD OF THE INVENTION

This invention relates to peptides which are useful in the context of cellular immunology. More particularly, the invention relates to peptides which bind to HLA molecules on the surface of cells. At least some of these peptides also induce the activation of cytolytic T cells when they are complexed with their partner HLA molecule. Also a part of the invention are the uses of these peptides in areas such as treating a cellular abnormality wherein the cells present complexes of HLA and peptides, identifying cells that express particular HLA molecules, e.g., HLA-A2, A3, A26, HLA-B7, B8, B15, B27, B35, B44 and B51, provoking a T cell response, determining the presence of particular T cells in a sample, as well as the cytolytic T cells themselves.

### BACKGROUND OF THE INVENTION

The study of the recognition or lack of recognition of cancer cells by a host organism has proceeded in many different directions. Understanding the field presumes some understanding of both basic immunology and oncology.

Early research on mouse tumors revealed that tumor cells displayed molecules which led to their rejection when transplanted into syngeneic animals. These molecules are “recognized” by T-cells in the recipient animal, and provoke a cytolytic T-cell response with lysis of the transplanted cells. This evidence was first obtained with tumors induced in vitro by chemical carcinogens, such as methylcholanthrene. The antigens expressed by the tumors and which elicited the T-cell response were different for each tumor. See e.g., Prehn et al., J. Natl. Canc. Inst. 18:769-778 (1957); Klein et al., Cancer Res. 20:1561-1572 (1960); Gross, Cancer Res. 3:326-333 (1943), Basombrio, Cancer Res. 30:2458-2462 (1970) for general teachings on inducing tumors with chemical carcinogens and differences in cell surface antigens. This class of antigens has come to be known as “tumor specific transplantation antigens” or “TSTAs.” Following the observation of the presentation of such antigens when induced by chemical carcinogens, similar results were obtained when tumors were induced in vitro via ultraviolet radiation. See Kripke, J. Natl. Canc. Inst. 53:333-1336 (1974).

While T-cell mediated immune responses were observed for the types of tumor described supra, spontaneous tumors were thought to be generally non-immunogenic. These were therefore believed not to present antigens which provoked a response to the tumor in the tumor carrying subject. See Hewitt et al., Brit. J. Cancer 33:241-259 (1976). The family of tum antigen presenting cell lines are immunogenic variants obtained by mutagenesis of mouse tumor cells or cell lines, as described by Boon et al., J. Exp. Med. 152:1184-1193 (1980), the disclosure of which is incorporated by reference. To elaborate, tum antigens are obtained by mutating tumor cells, which do not generate an immune response in syngeneic mice and will form tumors (i.e., “tum+” cells). When these tum+ cells are mutagenized, they are rejected by syngeneic mice, and fail to form tumors (thus “tum−”). See Boon et al., Proc. Natl. Acad. Sci. USA 74:272 (1977), the disclosure of which is incorporated by reference. Many tumor types have been shown to exhibit this phenomenon. See, e.g., Frost et al., Cancer Res. 43:125 (1983).

It appears that tum− variants fail to form progressive tumors because they initiate an immune rejection process. The evidence in favor of this hypothesis includes the ability of “tum⁻” variants of tumors, i.e., those which do not normally form tumors, to do so in mice with immune systems suppressed by sublethal irradiation, Van Pel et al., Proc. Natl. Acad. Sci. USA 76:5282-5285 (1979); and the observation that intraperitoneally injected tum− cells of mastocytoma P815 multiply exponentially for 12-15 days, and then are eliminated in only a few days in the midst of an influx of lymphocytes and macrophages (Uyttenhove et al., J. Exp. Med. 152:1175-1183 (1980)). Further evidence includes the observation that mice acquire an immune memory which permits them to resist subsequent challenge to the same tum variant, even when immunosuppressive amounts of radiation are administered with the following challenge of cells (Boon et al., Proc. Natl. Acad. Sci. USA 74:272-275 (1977); Van Pel et al., supra; Uyttenhove et al., supra). Later research found that when spontaneous tumors were subjected to mutagenesis, immunogenic variants were produced which did generate a response. Indeed, these variants were able to elicit an immune protective response against the original tumor. See Van Pel et al., J. Exp. Med. 157:1992-2001 (1983). Thus, it has been shown that it is possible to elicit presentation of a so-called “tumor rejection antigen” in a tumor which is a target for a syngeneic rejection response. Similar results have been obtained when foreign genes have been transfected into spontaneous tumors. See Fearon et al., Cancer Res. 48:2975-1980 (1988) in this regard.

A class of antigens has been recognized that are presented on the surface of tumor cells and are recognized by cytolytic T cells, leading to lysis. This class of antigens will be referred to as “tumor rejection antigens” or “TRAs” hereafter. TRAs may or may not elicit antibody responses. The extent to which these antigens have been studied, has been via cytolytic T cell characterization studies, in vitro, i.e., the study of the identification of the antigen by a particular cytolytic T cell (“CTL” hereafter) subset. The subset proliferates upon recognition of the presented tumor rejection antigen, and the cells presenting the tumor rejection antigens are lysed. Characterization studies have identified CTL clones which specifically lyse cells expressing the tumor rejection antigens. Examples of this work may be found in Levy et al., Adv. Cancer Res. 24:1-59 (1977); Boon et al., J. Exp. Med. 152:1184-1193 (1980); Brunner et al., J. Immunol. 124:1627-1634 (1980); Maryanski et al., Eur. J. Immunol. 124:1627-1634 (1980); Maryanski et al., Eur. J. Immunol. 12:406-412 (1982); Palladino et al., Cancer. Res. 47:5074-5079 (1987). This type of analysis is required for other types of antigens recognized by CTLs, including minor histocompatibility antigens, the male specific H-Y antigens, and the class of antigens referred to as “tum−” antigens, and discussed herein.

PCT application PCT/US92/04354, filed on May 22, 1992 assigned to the same assignee as the subject application, teaches a family of human tumor rejection antigen precursor coding genes, referred to as the MAGE family. Several of these genes are also discussed in van der Bruggen et al., Science 254:1643 (1991). It is now clear that the various genes of the MAGE family are expressed in tumor cells, and can serve as markers for the diagnosis of such tumors, as well as for other purposes discussed therein. See also Traversari et al., Immunogenetics 35:145 (1992) and De Plaen et al., Immunogenetics 40:360 (1994). The mechanism by which a protein is processed and presented on a cell surface has now been fairly well documented. A cursory review of the development of the field may be found in Barinaga, “Getting Some ‘Backbone’:How MHC Binds Peptides,” Science 257:880 (1992); also, see Fremont et al., Science 257:919 (1992); Matsumura et al., Science 257:927 (1992); Engelhard, Ann. Rev. Immunol. 12:181-207 (1994); Madden et al., Cell 75:693-708 (1993); Ramensee et al., Ann. Rev. Immunol. 11:213-244 (1993); Germain, Cell 76:287-299 (1994). These papers generally point to a requirement that the peptide which binds to an MHC/HLA molecule be nine amino acids long (a “nonapeptide”), and to the importance of the second and ninth residues of the nonapeptide. For H-2 kb, the anchor residues are positions 5 and 8 of an octamer, for H-2Db, they are positions 5 and 9 of a nonapeptide while the anchor residues for HLA-A1 are positions 3 and 9 of a nonamer. Generally, for HLA molecules, positions 2 and 9 are anchors.

Studies on the MAGE family of genes have now revealed that a particular nonapeptide is in fact presented on the surface of some tumor cells, and that the presentation of the nonapeptide requires that the presenting molecule be HLA-A1. Complexes of the MAGE-1 tumor rejection antigen (the “TRA”) leads to lysis of the cell presenting it by cytolytic T cells (“CTLs”).

U.S. Pat. No. 5,405,940 filed Aug. 31, 1992, and U.S. Pat. No. 5,571,711, disclose that there is a great deal of homology between various MAGE genes and the region of the MAGE-1 gene coding for the relevant nonapeptide. Indeed, these observations led to one of the aspects of the invention disclosed and claimed therein, which is a family of nonapeptides all of which have the same N-terminal and C-terminal amino acids. These nonapeptides were described as being useful for various purposes which includes their use as immunogens, either alone or coupled to carrier peptides. Nonapeptides are of sufficient size to constitute an antigenic epitope, and the antibodies generated thereto were described as being useful for identifying the nonapeptide, either as it exists alone, or as part of a larger polypeptide.

The preceding survey of the relevant literature shows that various peptides, usually eight, nine, or ten amino acids in length, complex with MHC molecules and present targets for recognition by cytolytic T cells. A great deal of research of melanoma, and melanoma antigens that are recognized by cytolytic T cells divides the antigens into three broad categories.

The first, which includes many of the antigens discussed, supra, (e.g., MAGE), are expressed in some melanomas, as well as other tumor types, and normal testis and placenta. The antigens are the expression product of normal genes which are usually silent in normal tissues.

A second family of melanoma antigens includes antigens which are derived from mutant forms of normal proteins. Examples of this family are MUM-1 (Coulie et al., Proc. Natl. Acad. Sci. USA 92:7976-7980 (1955)); CDK4 (Wölfel et al., Science 269:1281-1284(1955)); β-catenin (Robbins et al., J. Exp. Med. 183:1185-1192 (1996)); and HLA-A2 (Brandel et al., J. Exp. Med. 183:2501-2508 (1996)).

A third category, also discussed, supra, includes the differentiation antigens which are expressed by both melanoma and melanocytes. Exemplary are tyrosinase, gp100, gp75, and Melan A/Mart-1. See U.S. Pat. No. 5,620,886 incorporated by reference, with respect to Melan-A. See Wölfel et al., Eur. J. Immunol. 24:759 (1994) and Brichard et al., Eur. J. Immunol. 26:224 (1996) for tyrosinase; Kang et al., J. Immunol. 155:1343 (1995), Cox et al., Science 264:716 (1994) and Kawakami et al., J. Immunol. 154:3961 (1995) for gp 100, and; Wang et al., J. Exp. Med. 183:1131 (1996) for gp 75.

The molecule referred to as “NY-ESO-1”, as described in, e.g., U.S. Pat. No. 5,804,381, incorporated herein by reference, is recognized as one of the most immunogenic of tumor antigens. Nearly half of patients with advanced cancer express the antigen (Stockert et al., J. Exp. Med. 187:1349-1354 (1998)), and the expression is accompanied by both a strong CD4+ and a strong CD8+ T cell response. See Jäger et al., J. Exp. Med. 191:625-630 (2000); Jäger et al., J. Exp. Med. 167:265-270 (1998); Jäger et al., Proc. Natl. Acad. Sci. USA 97:4760-4765 (2000); Chen et al., J. Immunol., 165(2): 948-55 (2000). Peptides derived from the molecule which are HLA-A2 epitopes are known (Jäger et al., J. Exp. Med. 187:265-270 (1998)); and Wang et al., J. Immunol. 161:3598-3600 (1998), describes HLA-A31 binding epitopes. NY-ESO-1 also presents epitopes that bind to HLA-C molecules, such as HLA-Cw3 and HLA-Cw6 (Gnjatic et al., PNAS 97:10917-10922 (2000) incorporated herein by reference).

NY-ESO-1 has a homologous sequence to another tumor rejection antigen called LAGE-1 (Lethe et al. U.S. Pat. No. 5,811,519, incorporated herein by reference). It follows from what is known about the MAGE-A1/HLA-A1 and MAGE-A3/HLA-A1 peptides that the equivalent regions of LAGE-1 encoding the relevant nonapeptides would also present epitopes which bind with HLA-C molecules, such as HLA-Cw3 and HLA-Cw6. Disclosed herein is the discovery that NY-ESO-1 also presents epitopes that bind to many HLA molecules, e.g. HLA-A2, A3, A26, B7, B8, B15, B27, B35, B44 and B51, molecules. In some instances the peptides bind to more that one type of HLA molecule, which is not an unprecedented phenomenon, see, e.g., Schultz et al., Tissue Antigens. 57(2):103-9 (February 2001), Luiten et al., Tissue Antigens. 56(1):77-81 (July 2000);. Tomiyama et al., Eur J Immunol. 30(9):2521-30 (September 2000);. Thimme et al., J Virol., 75(8):3984-7 (April 2001) all incorporated herein by reference. These peptides and the ramifications of their discovery are a part of this invention. Also part of this invention are the compositions and methods for using these peptides. All facets of the invention are elaborated in the disclosure that follows.

### BRIEF DESCRIPTION OF THE FIGURES

FIG. 1. ELISPOT analysis of Ad2/ESO presensitized CD8+ NW1352 T cells against HLA-A3-matched allogeneic NW115 EBV-B cells pulsed with long overlapping NY-ESO-1 18-mer peptides.

FIG. 2. ELISPOT analysis of Ad2/ESO presensitized CD8+ NW1352 T cells against 7 NY-ESO-1 9-mer peptides binding to HLA-A3, that were pulsed onto T2.A3 cells as targets. The long NY-ESO-1 18-mer peptides p85-102 (SEQ ID NO: 5) and p91-108 (SEQ ID NO: 6) recognized in the first experiment were used as positive controls.

FIG. 3. ELISPOT analysis of Ad2/ESO presensitized CD8+ 1539 T cells against autologous monocyte-derived APC infected with wild-type or NY-ESO-1 adenovirus, or pulsed with NY-ESO-1- and SSX protein, or with a lysate of the NY-ESO-1+ melanoma cell line NW-MEL-38, or with NY-ESO-1 p94-102.

FIG. 4. ELISPOT analysis of NY-ESO-1 p94-102 presensitized CD8+ T cells from 4 NY-ESO-1 seropositive patients sharing the HLA-A3 allele. T2.A3 cells alone (−), or pulsed with NY-ESO-1 p94-102 (+) were used as target cells.

FIG. 5. Cytotoxicity of the T cell line NW1539-CTL-1/1 against NY-ESO-1 p94-102 pulsed T2.A3 cells, autologous vvESO transduced NW1539-EBV-B cells and NW1539-MEL-1 melanoma cells as assessed in a standard ⁵¹chromium release assay. No reactivity was found against T2.A3, untreated NW1539-EBV-B cells, and K562. Effector cells were used at effector to target ratios of 60 (checkered), 30 (striped), 10 (black), 1:1 (white).

FIG. 6. ELISPOT analysis of the CD8+ T cell NW1539-CTL-1/15 against T2 or T2.A3 cells alone, or pulsed with NY-ESO-1 p94-102 (+). The equal recognition of peptide pulsed T2 and T2.A3 cells suggests HLA-B51 as the restriction element for T cell recognition.

FIG. 7. ELISPOT analysis of NY-ESO-1 p94-102 presensitized NW1274 CD8+ T cells against PHA blasts of different HLA types generated from patients and healthy donors. PHA blasts were used alone (−), or pulsed with NY-ESO-1 p94-102 (+). Recognition of HLA-B51+ target cells NW1274 and NW1725 confirm the HLA-B51 restriction of T cell recognition.

FIG. 8. TNFα-release assays following stimulation of the CD8+ T cell lines NW923-IVS-1, NW1539-IVS-1, and NW1274-IVS-1 by COS-7 cell transfectants. TNF release was detected after stimulation with COS-7 cells co-transfected with the expression vector pcDNA3.1(−) containing NY-ESO-1 cDNA, and pcDNA1Amp containing HLA-B51 cDNA. No TNF release was detected after stimulation with COS-7 transfectants with HLA-A3 and NY-ESO-1, or with the T cell lines alone.

FIG. 9. ELISPOT analysis of unsensitized CD8+ selected NW1539 T cells against T2.A3 cells alone, or pulsed with NY-ESO-1 p94-102.

FIG. 10. ELISPOT assay with CD8+ T cells from patient UC-98 presensitized with NY-ESO-1 p80-109 and tested against histocompatible B-EBV targets pulsed with peptides at 10 μM (A and B) or at various concentrations (C).

FIG. 11. ELISPOT assay with CD8+ T cells from patient UC-98 presensitized with NY-ESO-1 p80-109 and tested against partially histocompatible B-EBV targets alone or pulsed with NY-ESO-1 p94-102. The complete class I haplotype from UC-98 is indicated at the top of the figure. Matching target alleles are shown in bold and italic print.

FIG. 12. ELISPOT assay with CD8+ T cells from patient UC-98 presensitized with NY-ESO-1 p80-109 and tested against histocompatible B-EBV targets pulsed with NY-ESO-1 peptides, or infected with vaccinia virus recombinant for NY-ESO-1 or wild-type.

FIG. 13. NY-ESO-1 amino acid sequence (SEQ ID NO: 14) and nucleotide sequence (SEQ ID NO: 15)

### SUMMARY OF THE INVENTION

This invention relates to peptides of NY-ESO-1, and their functional equivalents, which form complexes with HLA molecules, particularly HLA-A3, HLA-B35 and HLA-B51, and stimulate cytolytic T cells (CTLs) specific for complexes of the HLA and the peptides. The invention also relates to LAGE peptides that correspond to the peptides of this invention, e.g., p94-102 (SEQ ID NO: 1) of NY-ESO-1, particularly, MPFSSPMEA (SEQ ID NO: 13). This invention further relates to methods of using the peptides and their functional equivalents and to CTLs, which recognize the peptides in complex with an HLA molecule, and to methods of using antibodies, antibody fragments and soluble T cell receptors, as well as cells transduced to express the antibodies, antibody fragments or T cell receptors, which specifically bind to the peptides in complex with an HLA molecule.

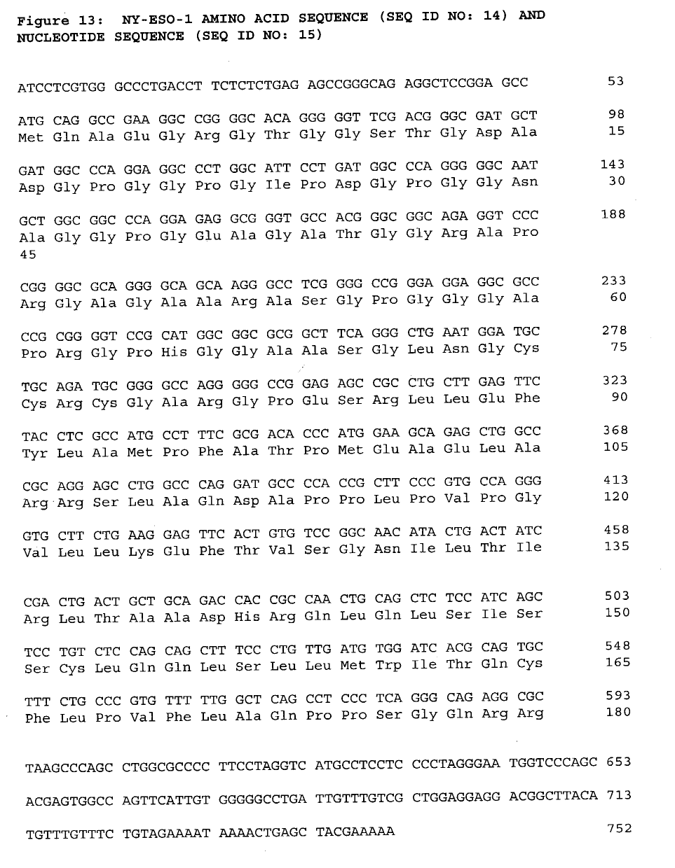

One embodiment of this invention is an isolated peptide which consists of between eight and eleven amino acids, which binds to an HLA molecule, particularly HLA-A2, A3, A26, B7, B8, B15, B27, B35, B44 and B51, more particularly an HLA-A3, HLA-B35 and/or an HLA-B51 molecule, and stimulates cytolytic T cells specific for complexes of a peptide consisting of an amino acid sequence selected from the group consisting of SEQ ID NO: 1 (NY-ESO-1 p94-102, Met Pro Phe Ala Thr Pro Met Glu Ala), SEQ ID NO: 2 (NY-ESO-1 p93-101, Ala Met Pro Phe Ala Thr Pro Met Glu), SEQ ID NO: 3 (NY-ESO-1 p108-116, Ser Leu Ala Gln Asp Ala Pro Pro Leu) and SEQ ID NO: 4 (NY-ESO-1 p91-99, Tyr Leu Ala Met Pro Phe Ala Thr Pro) and the HLA molecule, preferably HLA-A3, HLA-B35 and/or HLA-B51. Preferably the peptide comprises at least 8 contiguous amino acids of the sequence selected from the group consisting of SEQ ID NO: 1 (NY-ESO-1 p94-102), SEQ ID NO: 2 (NY-ESO-1 p93-101), SEQ ID NO: 3 (NY-ESO-1 p108-116) and SEQ ID NO: 4 (NY-ESO-1 p91-99). More preferably the isolated peptide consists of an amino acid sequence selected from the group consisting of SEQ ID NO: 1 (NY-ESO-1 p94-102), SEQ ID NO: 2 (NY-ESO-1 p93-101), SEQ ID NO: 3 (NY-ESO-1 p108-116) and SEQ ID NO: 4 (NY-ESO-1 p91-99). Most preferably the isolated peptide consists of the sequence set forth in SEQ ID NO: 1 (NY-ESO-1 p94-102) or SEQ ID NO: 2 (NY-ESO-1 p93-101). The NY-ESO-1 derived peptides referred to herein are identified by their corresponding position within NY-ESO-1 (SEQ ID NO: 14) FIG. 13.

Also an aspect of this invention is an isolated peptide which consists of between eight and eleven amino acids, which binds to an HLA molecule and stimulates cytolytic T cells specific for complexes of the peptide and the HLA molecule, e.g., HLA-A3, HLA-B35 or HLA-B51, molecule, wherein at least eight contiguous amino acids of said peptide consist of a sequence of at least eight contiguous amino acids of the LAGE peptide MPFSSPMEA (SEQ ID NO: 13). Preferably the peptide consists of the sequence MPFSSPMEA (SEQ ID NO: 13).

Also an embodiment of this invention is a composition comprising the isolated peptides of this invention and a suitable carrier. Preferably the composition comprises a peptide consisting of the amino acid sequence set forth in SEQ ID NO: 1 or SEQ ID NO: 2. The composition may further comprise an adjuvant. The adjuvant may be any pharmaceutically acceptable adjuvant available in the art, e.g., a cytokine. Preferably the cytokine is granulocyte-macrophage colony-stimulating factor (GM-CSF) or interleukin-12 (IL-12).

A further aspect of this invention are nucleic acid molecules which encode the peptides of this invention. Preferably the nucleic acid molecules encode a peptide consisting of SEQ ID NO: 1 (NY-ESO-1 p94-102), SEQ ID NO: 2 (NY-ESO-1 p93-101), SEQ ID NO: 3 (NY-ESO-1 p108-116) and SEQ ID NO: 4 (NY-ESO-1 p91-99). More preferably the nucleic acid molecule encodes a peptide that consists of the sequence set forth in SEQ ID NO: 1 (p94-102) or SEQ ID NO: 2 (p93-101). Oligonucleotides encoding the peptides of this invention can be easily prepared using methods that are standard in the art.

Expression vectors comprising the isolated nucleic acid molecule of this invention in operable linkage with a promoter are also contemplated herein. The expression vector may be any that is known in the art, e.g., a plasmid, a cosmid, a bacteriophage or a viral vector. The nucleic acid molecule may be operatively linked to any promoter known in the art. Those of skill in the art are well-versed in recombinant DNA technologies and would appreciate that many different promoters are available and the choice of promoter should be one that is compatible with a particular host environment. The nucleic acid molecules and expression vectors of this invention are also useful for inducing an immune response in a subject in need thereof wherein the vectors are administered to the subject in an amount sufficient to induce an immune response, e.g., the production of antibodies that bind the peptide or NY-ESO-1 or the stimulation of CD8+ T cells specific for a complex of HLA and the peptide of this invention.

Host cells transformed or transfected with the nucleic acid molecules and expression vectors of this invention are also contemplated. Such host cells may be prokaryotic, for example, E. coli, or eukaryotic., e.g, mammalian cells such as e.g., mouse, rat, hamster, monkey or human cells, avian cells, such as, e.g., chicken or duck, or insect or plant cells.

Another aspect of this invention are cytolytic T cells (CTLs) specific for a complex of an HLA molecule and a peptide selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100). Preferably the isolated cytolytic T cell is specific for a complex of an HLA molecule, e.g, HLA-A2, A3, A26, B7, B8, B15, B27, B35, B44, B51, particularly HLA-A3, HLA-B35 and HLA-B51, and a peptide which consists of the amino acid sequence set forth in SEQ ID NO: 1 or SEQ ID NO: 2.

This invention also relates to methods for monitoring or detecting CTLs specific for a complex of the peptides of this invention, particularly SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100), and an HLA molecule, e.g., HLA-A2, A3, A26, B7, B8, B15, B27, B35, B44 or B51 particularly an HLA-A3, and HLA-B35 or HLA-351 molecule. One method for detecting a CTL specific for a such complexes may comprise: (a) contacting a cytolytic T cell-containing sample with a composition comprising cells presenting a complex of the HLA molecule, e.g. an HLA-A3 molecule, an HLA-B35 molecule or an HLA-B51 molecule, and the isolated peptide and (b) determining if a CTL in the CTL containing sample recognizes the cells presenting the complex of HLA and the isolated peptide, wherein recognition of the complex is indicative of a CTL specific for a complex of the HLA molecule and the isolated peptide.

The cells which express complex on their surfaces my be transfected or transformed with a nucleic acid molecule which encodes the peptide. Alternatively, the cells may be transfected with a nucleic acid molecule that encodes the peptide and transfected with a nucleic acid molecule that encodes the HLA molecule, e.g. an HLA-A3, HLA-B35 or an HLA-B51 molecule. The cells may also be transfected with a nucleic acid molecule that encodes both the HLA molecule and the peptide.

Another aspect of this invention are polytopes comprising one or more of the peptides of this invention. Polytopes are groups of two or more potentially immunogenic or immune stimulating peptides, which can be joined together in various ways, to determine if this type of molecule will stimulate and/or provoke an immune response. Preferably the polytope comprises a peptide consisting of the amino acid sequence set forth in SEQ ID NO: 1 or SEQ ID NO: 2, or the polytope comprises a peptide consisting of the amino acid sequence set forth in SEQ ID NO: 1 and a peptide consisting of the amino acid sequence set forth in SEQ ID NO: 2. The polytopes of this invention may be used to induce an immune response in a subject in need thereof. For example, the subject may be one having a disorder characterized by the expression of HLA/peptide complexes on cell surfaces, e.g., HLA-A3, B35 or B51 and a peptide consisting of the sequence set forth in SEQ ID NO: 1, 2, 3, 4, 5, 6, 7, 8 or 13. The immune response may be the production of antibodies specific for the peptide, the peptide complex or NY-ESO-1 or the immune response may be the stimulation of CD8+ T cells specific for the HLA/peptide complexes of this invention.

Also a part of the invention are so-called “minigenes,” i.e., nucleic acid molecules consisting of a nucleotide sequence that encodes the peptide of interest. The peptides of this invention are of a length that permits simple construction of all degenerate nucleotide sequences that encode the epitope of interest. These coding sequences can be made a part of an extended “polytopic” sequence, using methods well known in the art, and can be incorporated into coding vectors where the minigene or genes of interest are operably linked to a promoter, for expression in a host cell.

The minigenes can also be used in concert with nucleotide sequences that encode an MHC molecule of interest, such as HLA-A3, B35 or B51, coding sequence. The two sequences can constitute part of a single vector, or a pair of vectors, which are then used in a kit or some other combination that permits the skilled artisan to use them to stimulate a T cell response, and so forth.

Similarly, one can envision treatment methodologies which employ dendritic cells, pulsed with the peptides of this invention, as well as cells which have been treated so as to present relevant complexes on their surfaces, see for example U.S. Pat. No. 6,251,603 issued Jun. 26, 2001 incorporated herein by reference. Such cells may be transformed or transfected with a tumor rejection antigen precursor TRAP gene, e.g., NY-ESO-1, or a TRAP “minigene” or “minigenes”, which encodes only relevant MHC binding peptides such as tumor rejection antigens, and/or may be transfected or transformed with a relevant MHC-molecule encoding sequence, such as HLA-A2, A3, A26, B7, B8, B15, B27, B35, B44, B51 and so forth. If appropriate, such cells may be irradiated prior to administration.

This invention also relates to the isolated nucleic acid molecules that encode the polytopes of this invention, nucleic acid molecules in operable linkage with a promoter and to expression vectors comprising those nucleic acid molecules in operable linkage with a promoter. The expression vector may be any that is known in the art, e.g., a viral vector, a plasmid or a cosmid. The vectors may be used to induce an immune response in a subject in need thereof, e.g., a subject having a disorder characterized by the expression of the HLA/peptide complexes of this invention on cell surfaces, by administering a composition comprising the vector in an amount sufficient to induce an immune response

Also an embodiment of this invention are host cells transformed or transfected with the isolated nucleic acid molecule or expression vectors of this invention. Host cells may be any that are used routinely in the art, bacterial cells, e.g., E. coli, insect cells, mammalian cells, e.g., mouse, hamster, rat, cat, dog, horse, pig, monkey or human, avian cells, e.g., chicken, duck or goose, plant cells, e.g. soy bean, tobacco, rice, wheat or corn. Methods for the transformation or transfection of particular host cells are well known in the art and need not be described in detail herein.

Multicomponent complexes, tetramers, which are useful in the analysis of T cell populations are also an aspect of this invention. The construction of such tetramers is disclosed in U.S. Ser. No. 09/275,993 filed Mar. 24, 1999 incorporated herein by reference, see also Dunbar et al., Curr. Biol. 8:4132-416 (1998) incorporated herein by reference. The tetramers of this invention comprise HLA molecules, particularly HLA-A2, A3, A27, B7, B8, B15, B27, B35, B44 and B51, more particularly HLA-A3, HLA-B35 or HLA-B51 molecules, β2 microglobulin and the peptides of this invention. For example, an tetramer may comprise an HLA, e.g. HLA-A3, HLA-B35 or HLA-B51, a β2 microglobulin, a peptide comprising an amino acid sequence selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100), a biotin molecule and a binding partner, e.g., avidin or strepavidin. The HLA molecule and β2 microglobulin refold into a complex which is then biotinylated with biotin holoenzyme synthase and then combined with labeled streptavidin or labeled avidin to produce tetrameric structures. The tetrameric structures are mixed with the particular peptides and the multicomponent complex (“tetramer”) is then used to identify CTL cells that are specific for the complex of HLA and peptide. The tetramers in some circumstances may also stimulate the CTL cells to release cytokines or proliferate. Preferably the tetramer comprises a peptide that consists of an amino acid sequence set forth in SEQ ID NO: 1 or SEQ ID NO: 2.

Compositions comprising the tetramers are also part of this invention, as are methods for using the tetramers to identify CTLs which recognize specific HLA/peptide complexes and to methods of stimulating the CTLs with the tetramers. The composition may also comprise a carrier, and/or an adjuvant. The carrier and adjuvant may be any that are pharmaceutically acceptable and routinely used in the art. E.g. the carrier may be DMSO and the adjuvant may be GM-CSF or IL-12.

The peptides of this invention either alone or in complex with an HLA molecule, e.g. A2, A3, A26, B7, B8, B15, B27, B35 B44 or B51, particularly HLA-A3, HLA-B35 or HLA-B51 are useful for inducing an immune response in a subject, e.g., the production of antibodies or the stimulation of CD8+ cells. Methods for inducing an immune response in a subject may comprise administering a composition comprising a peptide of this invention or complexes of the peptide and MHC molecules, e.g., cells presenting a complex of peptide and MHC on their surfaces, to a subject wherein the amount of the peptide or complex administered is sufficient to induce an immune response, either humoral or cellular.

If cells expressing the peptide are administered to a subject they should be cells that do not have harmful effects on the subject, e.g., the cells may be irradiated such that they do not proliferate in the subject but still display the complex of HLA and peptide and the cells should be non-tumorigenic. The cells may autologous and may be transfected with a nucleic acid molecule that encodes the peptide or may be transfected with a nucleic acid molecule that encodes the HLA molecule, e.g., HLA-A3, HLA-B35 or HLA-B51. The cells may also be transfected with a nucleic acid molecule that encodes both the peptide and the HLA molecule.

This invention also relates to a method for treating a subject with a disorder characterized by the presence of complexes of an HLA molecule, particularly an HLA-A3, HLA-B35 and/or HLA-B51 molecule, and a peptide of this invention, particularly a peptide having an amino acid sequence selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100). presented on cell surfaces. One embodiment of the invention is to administer the peptide to the subject in an amount that is sufficient to induce an antibody response or in an amount sufficient to stimulate CD8+ T lymphocytes specific for complexes of an HLA and the peptide in sufficient numbers to alleviate the disorder. The method is particularly useful for a subject who has cancer cells that express NY-ESO-1 and particularly a subject whose cells express HLA-A3, HLA-B35 and/or HLA-B51 as well. The generation and proliferation of the CTLs may be monitored by any means known in the art.

This invention further relates to a method for treating a subject with a disorder characterized by the presence of complexes of an HLA molecule, particularly HLA-A3, HLA-B35 and/or HLA-B51, and a peptide of this invention, particularly one selected from the group consisting of SEQ ID NO: 1 (p94-102), SEQ ID NO: 2 (p93-101), SEQ ID NO: 3 (p108-116), and SEQ ID NO: 4 (p91-99) on cell surfaces by administering an amount of cytolytic T cells, which are specific for the complexes of the HLA molecule and the peptide, to the subject wherein the amount is sufficient to alleviate the disorder.

Another embodiment of this invention is a method for inducing an immune response in a subject in need thereof, e.g., a subject having a disorder characterized by the presentation of complexes of HLA and peptide, e.g., HLA-A3, B35 or B51 and a peptide consisting of the sequence set forth in SEQ ID NO: 1, 2, 3, 4, 5, 6, 7, 8, or 13 by administering the peptides, proteins, tetramers, polytopes, nucleic acid molecules and vectors comprising the nucleic acid molecules of this invention to the subject in an amount sufficient to alleviate the disorder. The immune respones may be e.g., the induction of antibodies specific for the peptides, peptide analogs, proteins, e.g., NY-ESO-1, or HLA/peptide complexes of this invention or the stimulation of CD8+ cells that are specific for complexes of the HLA and peptide described herein.

Also a part of this invention are peptide analogs, e.g., functional equivalents of the peptides consisting of the amino acid sequences set forth in SEQ ID NO: 1 (p94-102), SEQ ID NO: 2 (p93-101), SEQ ID NO: 3 (p108-116) and SEQ ID NO: 4 (p91-99). The peptide analogs are isolated peptides which, when complexed to an MHC molecule, are recognized by the cytolytic T cells that recognize a complex of an HLA-A3 molecule, an HLA-B35 molecule or HLA-B51 molecule, and a peptide selected from the group consisting of SEQ ID NO: 1 (p94-102), SEQ ID NO: 2 (p93-101), SEQ ID NO: 3 (p108-116) and SEQ ID NO: 4 (p91-99). Such analogs may be isolated from a combinatorial library of peptides that are from 8-11 amino acids in length and comprise naturally occurring or synthetic amino acids. The combinatorial library may be screened for peptides that bind to HLA-A3, HLA-B35 and/or HLA-B51 and those peptides may be assayed for their ability, when complexed with the HLA, to be recognized by cytolytic T cells that recognize a complex of the HLA molecule and a peptide selected from the group consisting of SEQ ID NO: 1 (p94-102), SEQ ID NO: 2 (p93-101), SEQ ID NO: 3 (p108-116) and SEQ ID NO: 4 (p91-99).

Alternatively, rather than isolating the peptide analogs from a combinatorial library of random peptides, a plurality of peptide derivatives may be systematically prepared based on the known sequences of the peptides of this invention. Peptides selected from the group consisting of SEQ ID NO: 1 (p94-102), SEQ ID NO: 2 (p93-101), SEQ ID NO: 3 (p108-116) and SEQ ID NO: 4 (p91-99) may serve as the basis for the generation of a combinatorial library of peptide derivatives which may be screened for peptides which, when complexed to an MHC molecule, are recognized by cytolytic T cells which recognize a complex of an HLA molecule, e.g. HLA-A3, HLA-B35 or HLA-B51 molecule, and a peptide selected from the group consisting of SEQ ID NO: 1 (p94-102), SEQ ID NO: 2 (p93-101), SEQ ID NO: 3 (p108-116) and SEQ ID NO: 4 (p91-99). Such libraries may be generated by substituting one or more to the residues of the known peptides or by modifying one or more of the residues of the peptides as described in detail infra.

### DETAILED DESCRIPTION OF THE INVENTION

This invention relates to peptides of NY-ESO-1, particularly peptides consisting of an amino acid sequence selected from the group consisting of SEQ ID NO: 1 (p94-102), SEQ ID NO: 2 (p93-101), SEQ ID NO: 3 (p108-116) and SEQ ID NO: 4 (p91-99). The peptides may be produced synthetically using standard techniques known in the art e.g., manufactured by good manufactorial practice guidelines (Multiple Systems, San Diego), or may be produced recombinantly using an expression vector that comprises a nucleic acid molecule that encodes the peptide in operable linkage with a promoter.

The invention also relates to peptide analogs, i.e., peptides that do not have the sequence set forth in SEQ ID NO: 1 (p94-102), SEQ ID NO: 2 (p93-101), SEQ ID NO: 3 (p108-116) and SEQ ID NO: 4 (p91-99) and yet form complexes with HLA molecules, particularly HLA-A3, HLA-B35 and/or HLA-B51 molecules, and stimulate cytolytic T cells (CTLs) specific for complexes of the HLA and a peptide consisting of an amino acid sequence selected from the group consisting of SEQ ID NO: 1 (p94-102), SEQ ID NO: 2 (p93-101), SEQ ID NO: 3 (p108-116) and SEQ ID NO: 4 (p91-99). The peptides may be, e.g., from 8-11 amino acids in length and preferably comprise at least 8 contiguous amino acids of the sequences set forth in SEQ ID NO: 1 (p94-102), SEQ ID NO: 2 (p93-101), SEQ ID NO: 3 (p108-116) or SEQ ID NO: 4 (p91-99).

The peptide analogs of this invention may be modified with protective groups at one or both ends, or one or more peptide bonds may be replaced with non-peptide bonds to make them less susceptible to proteolytic cleavage than the non-modified peptides. For example, one or more peptide bonds may be replaced with an alternative type of covalent bond (e.g., a carbon-carbon bond or an acyl bond). Where proteolytic degradation of the peptide following administration to a subject is a problem, the sensitive peptide bonds may be replaced to increase the stability of the peptide and increase its usefulness as a therapeutic. Peptidase sensitive bonds may be determined by standard methods, see for example, application No. 60/290,646 incorporated herein by reference. Methods of incorporating various types of bonds into peptides, are well known in the art and need not be described in detail here. Peptide analogs may also be generated by incorporating amino-terminal or carboxyl terminal blocking groups such as t-butyloxycarbonyl, acetyl, alkyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl, thereby rendering the peptide analog or mimetic less susceptible to proteolysis. Non-peptide bonds and carboxyl- or amino-terminal blocking groups can be used singly or in combination to render the peptide analog less susceptible to proteolysis than the corresponding peptide. Peptide with modifications like β-a.a. (β-amino acid), d-a.a, αMe-a.a. or NMe-a.a. may be synthesized by incorporation of a corresponding modified amino acid. Many modified amino acids are commercially available (see, e.g., Bachem A G, Budendorf, Switzerland (NMe-a.a.), Fluka Chemie GmbH, Bush, Switzerland (α-a.a.), Acros Organic France, Noisy-Le-Grand, France (αMe-a.a) Me amino acid are often sold in a racemic form, and as a consequence, peptides with Me modification may also be produced in a racemic form.

The peptides of this invention may comprise a modification such as for example a methylation of an carboxy group (Me-peptide) methylation of a nitrogen engaged in peptidic bond formation (NMe-peptide), acetylation of a terminal nitrogen (acetyl peptide), amidation of a terminal carboxylic group (amide-peptide), reduced bond (Ψ1-2(CH2-NH), β-amino acid (aa), e.g., β-alanine, β-glutamic acid, D-amino acid (d-aa), hydroxylation of a terminal nitrogen (NOH-peptide), retro-inverso peptide bond (Ψ1-2(CO-NH2), and cyclic amino acid, e.g., pyro-glutamic a.a.. See e.g., U.S. patent application Ser. No. 09/114,002 filed Jul. 10, 1998 and incorporated herein by reference for examples of methods for modifying peptides to increase their resistance to proteolytic cleavage and for assays of peptide stability. A reduced bond Ψ(CH2-NH) can be formed by the reductive alkylation of a free amino group with a Fmoc protected amino aldehyde performed according to the method developed by Fehrentz and Castro (Fehrentz and Castro, Synthesis, 676-678 (1983) incorporated herein by reference). Peptides may be purified by reverse-phase high-pressure liquid chromatography (RP-HPLC) on a C8 column (Aquapore (Brownlee)). Identity of the purified peptides can be confirmed by mass spectrometry using electrospray ionisation (ESI-MS). Peptide stock solutions may be adjusted to appropriate peptide concentration in, e.g., 100% dimethylsulfoxyde (DMSO), and stored at −20° C. N-terminal-hydroxy peptides can be synthesized according to methods disclosed in e.g., Bianco et al., J. Peptide Sci., 4:471-478 (1998), Guichard et al., J. Med. Chem., 39: 2030-2039 (1996) and Dürr et al., Angew. Chem. Int. Ed. Engl., 31(6): 785-787 (1992), all incorporated herein by reference. The peptides of this invention may be synthesized by any method known in the art, e.g. the solid-phase method using the Fmoc chemistry and DIPC/HOAt (N,N diispropylcarbodiimmide/N-hydroxyazabenzotiazole) coupling procedure.

Preferably the peptide analogs of this invention display very similar HLA binding and CTL recognition compared to the non-modified peptide. Preferably the HLA is an HLA-A3, HLA-B35 or an HLA-B51 molecule.

Combinatorial libraries of derivatives of peptides having an amino acids of the sequences set forth in SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100), particularly SEQ ID NO: 1 may be generated by the systematic substitution, deletion or modification of one or more amino acids of the sequences set forth in SEQ ID NO: 1 (p94-102), SEQ ID NO: 2 (p93-101), SEQ ID NO: 3 (p108-116) and SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100). For example, the residues at positions 2 and 9 in the foregoing sequences may be held constant while one or more of the remaining residues are substituted, deleted or modified. For instance, for a library of peptides based upon SEQ ID NO: 1, the amino acid at position two of the starting peptide would be a proline and the amino acid at position 9 would be an alanine. These two amino acids would be held constant while one or more amino acids would be systematically substituted, deleted and/or modified.

The peptides and libraries may be constructed using well known methods. See, e.g., Merrifield, R. B., “Solid phase peptide synthesis. I. The synthesis of a tetrapeptide”, J. Am. Chem. Soc. 85:2149-2154 (1963); M. Bodanszky, “Principles of Peptide Synthesis”, Springer-Verlag p. 21-27 (1984); Jung et al., “Multiple Peptide Synthesis Methods and Their Applications” Angew. Chem. Int. Ed. Engl., vol. 31, No. 4, pp. 367-383 (April 1992); Janda, K. D., “Tagged versus untagged libraries: Methods for the generation and screening of combinatorial chemical libraries”, Proc. Natl. Acad. Sci. USA, vol. 91, pp. 10779-10785 (November 1994), and Barbas et al., Phage Display: A Laboratory Manual, Cold Spring Harbor Laboratory Press, Cold Spring Harbor N.Y. (2001) (all incorporated herein by reference). For example, oligonucleotides that encode derivatives of the peptides may be generated by synthesizing oligonucleotides using mixtures of two or more of the four nucleoside triphosphates rather than pure preparations of the nucleoside triphosphates. The mixtures of nucleosides introduce changes into the oligonucleotide sequence and these changed oligonucleotides encode peptide derivatives that differ in only a few residues from the initial peptide. Peptide derivatives may also be prepared, for example, by using a pIII or a pVIII based peptides on phage system, and combinatorial libraries can be screened to identify a phage that presents a peptide analog that binds to an MHC molecule and the DNA of the phage screened to determine the sequence of the peptide analog displayed on the surface of the phages. Additional methods for generating libraries of peptides are also disclosed in, e.g., U.S. Pat. No. 5,932,546, incorporated herein by reference.

The derivatives of the peptides may be generated by substituting one or more amino acids in SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120), and SEQ ID NO: 8 (p92-100) with a different naturally occurring amino acid or a synthetic amino acid analog to generate a plurality of peptide derivatives. Alternatively, one or more amino acids may be chemically modified, e.g., as described supra, to generate a plurality of chemically modified peptide derivatives. The derivatives may then be screened for analogs of the peptides consisting of the sequence set forth in SEQ ID NO: 1, 2, 3, 4, 5, 6, 7 or 8, preferably SEQ ID NO: 1, 2, 3 or 4. Preferably the chemically modified peptide analogs are more stable than the corresponding non-modified peptides but are still recognized by CTLs that recognize a complex of an HLA particularly HLA-A3, HLA-B35 or HLA-B51 and a peptide consisting of an amino acid sequence selected from the group consisting of SEQ ID NO: 1, 2, 3, 4, 5, 6, 7, and 8.

A combinatorial library of the peptide derivatives may be screened for analogs that have the following characteristics: a peptide that does not consist of the sequence set forth in SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120), and SEQ ID NO: 8 (p92-100), and yet binds to an MHC molecule, preferably HLA-A2, A3, A26, A27, B35, B44 or B51, more preferably an HLA-A3, HLA-B35, or HLA-B51 molecule, and when bound to the MHC molecule they form a complex that is recognized by a cytolytic T cell that recognizes a complex of the HLA molecule, preferably HLA-A3 or HLA-B35 or HLA-B51, or both, and a peptide selected from the group consisting of SEQ ID NO: 1 (p94-102), SEQ ID NO: 2 (p93-101), SEQ ID NO: 3 (p108-116) and SEQ ID NO: 4 (p91-99) preferably SEQ ID NO:1. Such peptide analogs are useful in the methods of this invention, e.g., for stimulating CTL cells that recognize cells presenting a complex of an HLA molecule e.g. HLA-A2, A3, A26, B7, B8, B15, B27, B35 B44 or B51 and a peptide selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100).

The peptides of this invention may be incorporated into polytopes. Two or more peptides of this invention can be joined together directly, or via the use of flanking sequences. See Thompson et al., Proc. Natl. Acad. Sci. USA 92(13): 5845-5849 (1995), teaching the direct linkage of relevant epitopic sequences. The use of polytopes as vaccines is well known. See, e.g. Gilbert et al., Nat. Biotechnol. 15(12): 1280-1284 (1997); Thomson et al., supra; Thomson et al., J. Immunol. 157(2): 822-826 (1996); Tam et al., J. Exp. Med. 171(1): 299-306 (1990), all of which are incorporated by reference. The Tam reference in particular shows that polytopes, when used in a mouse model, are useful in generating both antibody and protective immunity. Further, the reference shows that the polytopes, when digested, yield peptides which can be and are presented by MHCs. Tam demonstrates this by showing recognition of individual epitopes processed from polytope ‘strings’ via CTLs. This approach can be used, e.g., in determining how many epitopes can be joined in a polytope and still provoke recognition and also to determine the efficacy of different combinations of epitopes. Different combinations may be ‘tailor-made’ for patients expressing particular subsets of tumor rejection antigens. These polytopes can be introduced as polypeptide structures, or via the use of nucleic acid delivery systems. To elaborate, the art has many different ways available to introduce DNA encoding an individual epitope, or a polytope such as is discussed supra. See, e.g., Allsopp et al., Eur. J. Immunol. 26(8); 1951-1959 (1996), incorporated by reference. Adenovirus, pox-virus, Ty-virus like particles, plasmids, bacteria, etc., can be used. One can test these systems in mouse models to determine which system seems most appropriate for a given, parallel situation in humans. They can also be tested in human clinical trials.

The peptides of this invention either alone or in complex with an HLA molecule, particularly HLA-A3, HLA-B35 or HLA-B51, are useful for inducing an immune response in a subject, either humoral or cellular. The peptides may induce an immune response that may be either protective or therapeutic. The methods for inducing an immune response in a subject comprise administering a composition comprising an amount of a peptide of this invention in an amount that is sufficient to induce an immune response. The composition may comprise complexes of the inventive peptides and an HLA molecule or e.g. the composition may comprise cells which present the peptides in complex with an HLA molecule. Methods for immunizing a subject with a composition are well known in the art, see e.g., Jager et al. PNAS 97(9): 12198-12203 (Oct. 24, 2000) incorporated herein by reference.

If cells presenting a complex the HLA and the inventive peptides are administered to a subject, they should be cells that do not have harmful effects on the subject, e.g., the cells may be irradiated to insure they do not proliferate or the cells may be non-tumorigenic. The cells expressing the HLA/peptide may be autologous and may be transfected with a nucleic acid molecule that encodes the peptide. If the presenting cells do not naturally express a particular HLA molecule, e.g. HLA-A2, A3, A26, B7, B8, B15, B27, B35, B44 or B51, the cells may also be transfected with a nucleic acid molecule that encodes the HLA. Alternatively the presenting cells may be transfected with a nucleic acid molecule that encodes both the peptide and HLA molecule. Likewise the polytopes and peptide analogs of this invention, which form complexes with the MHC that are recognized by the CTLs that recognize complexes of the HLA and a peptide having an amino acid sequence of SEQ ID NO: 1, 2, 3 or 4, may also be used to induce an immune response in a subject, preferably a subject with a disorder characterized by the presentation of an HLA and a peptide having the amino acid sequence set forth in SEQ ID NO: 1, 2, 3 or 4. For example the subject may have a cancer, wherein the cancer cells express the HLA/peptide complex on their surfaces.

Compositions which comprise the peptides, peptide analogs, polytopes, tetramer complexes or CTLs of this invention may further comprise a carrier, and/or an adjuvant. The carrier and adjuvant may be any that are routinely used in the art and are pharmaceutically acceptable. E.g. the carrier may be DMSO and the adjuvant may be GM-CSF or IL-12. A pharmaceutically or therapeutically acceptable or suitable carrier is a carrier medium is preferably one that does not interfere with the effectiveness of the biological activity of the active components and which is not toxic to the subject.

The immunogenicity of peptides may be assayed by a variety of methods routinely used by one of skill in the art, see e.g., Jager et al. PNAS, 97(9):4760-4765 (Apr. 25, 2000) incorporated herein by reference and Jager et al.,(Oct. 24, 2000) supra. The immunogenicity may be assayed in vitro by their ability to stimulate peripheral blood lymphocytes that are positive for HLA-A2, A3, A26, B7, B8, B15, B27, B35, B44 or B51. The peripheral blood lymphocytes are preferably from healthy HLA-A3, HLA-B35 or HLA-B51 donors. Many assays are known in the art for determining if a CTL cell is stimulated by a cell that is presenting an HLA/peptide complex, e.g., ELISPOT, cytotoxicity assays and DTH assays (Jager et al. (April 2000) supra and Jager et al. (October 2000) supra). When a CTL cell recognizes a particular HLA-peptide complex it is activated and this activation is manifested by, e.g., CTL proliferation, lysis of cells presenting a complex of HLA-A3, HLA-B35 or HLA-B51 and the peptide by the activated CTL, and release of cytokines by the activated CTLs. The release of cytokines may be assayed by e.g., ELISPOT, as described infra. The activation of the CTLs may also be detected by admixing the CTL cell containing sample with a tetramer, as described supra, that is composed of a the peptide of this invention, an HLA molecule, particularly an HLA-A3, HLA-B35 or HLA-B51 molecule, a β2-microglobulin and biotin, which may then be labeled with a molecule such as e.g. avidin or streptavidin (see U.S. application Ser. No. 09/725,993, incorporated herein by reference, for a description of the generation and use of such tetramers.) Preferably the tetramer comprises HLA-A3, HLA-B35 or HLA-B51, a peptide of this invention, particularly a peptide consisting of the sequence set forth in SEQ ID NO: 1, 2, 3 or 4, and biotin. Such tetramers, compositions comprising the tetramer complexes and methods for their use are also aspects of this invention.

In a method for indentifying T cells, such as CD8+ T cells which are specific to a peptide/MHC complex, where the peptide derives from a protein of interest, a sample believed to contain relevant CD8+ cells is contacted to an antigen presenting cell, such as a dendritic cell, which has been infected with a first viral vector that encodes the protein of interest. Following this contact, the CD8+ cells are then contacted with a second population of antigen presenting cells which have been infected with a second viral vector which also encodes the protein of interest, where the second viral vector is different from the first viral vector. One benefit that is believed to be derived from this approach is that any immune response can be more refined in that it is targeted to the antigen rather than any aspect of the viruses. In a preferred embodiment the first viral vector is an adenovirus vector, preferably one that is non replicative, and the second vector is a vaccinia vector. It will be understood, however, that these may be reversed, and that only one of these two choices can be used, in combination with a second virus that differs from one of these two choices.

The method requires an antigen presenting cell, such as a dendritic cell, or some other cell type capable of presenting complexes of an MHC or HLA molecule and a peptide on its surface. In practice, the method preferably involves the use of autologous cells, i.e., antigen presenting cells and CD8+ T cells from the same patient, but the methodology can be carried out with allogeneic cells as well. Use of the method, permits one to identify epitopes that are restricted by their presenting MHC/HLA molecule. As shown herein, the method permit identification of peptides which bind to HLA molecules such as HLA-A3, HLA-B35 or HLA-B51 molecules including, but not being limited to the peptides defined by SEQ ID NOS: 1, 2, 3 and 4. These peptides can be used, e.g., to stimulate production of cytolytic T cells specific for complexes of the HLA molecule and the peptide to identify cells presenting the HLA molecule, and so forth. The peptides can be used therapeutically as, e.g., the single peptide component of a formulation designed to enhance an immune response, or as one of a plurality of more than one peptide. Such compositions may include an additional component, such as an adjuvant.

The methods of this invention are particularly useful for detecting the presence of, and monitoring the proliferation of, CTLs in a cell sample taken from a subject having a disorder associated with the presentation of complexes of HLA and a peptide having an amino acid sequence set forth in SEQ ID NO: 1, 2, 3 or 4, e.g., a cancer, e.g., melanoma, wherein the CTLs are specific for the complex an HLA molecule, e.g. HLA-A2, A3, A26, B7, B8, B15, B27, B35, B44 or B51, particularly HLA-A3, HLA-B35 or HLA-B51 and a peptide having an amino acid sequence set forth in SEQ ID NO: 1, 2, 3 or 4. The subject may be monitored for the generation and proliferation of CD8+ T lymphocytes by assaying cell samples from the subject at various times to determine the number of CTLs that are specific for a particular complex of the HLA and peptide and to determine if that number is increasing or decreasing over time. In particular, the peptide of this invention may be administered to the subject and then the subject may be monitored for a response by CTLs and for their subsequent proliferation. The peptide may be administered to the subject in a form where it is not in complex with an HLA molecule or it may be administered in complex with the HLA molecule, particularly HLA-A3, HLA-B35 or HLA-B51. The peptide may be administered with any pharmaceutically suitable carrier, and may also be administered with a pharmaceutically acceptable adjuvant, e.g., GM-CSF or IL-12. In one embodiment intact cells or cell parts that present the complex of HLA and peptide on their surface may be administered to the subject in a pharmaceutically acceptable carrier. The method is particularly useful for a subject who has cancer cells that express NY-ESO-1 and particularly one whose cells express HLA-A3, HLA-B35 or HLA-B51 as well.

Also a part of this invention are antibodies, e.g., polyclonal and monoclonal, and antibody fragments e.g., single chain Fv, Fab, diabodies, etc. and T cell receptors, that specifically bind the peptides or HLA/peptide complexes disclosed herein. Preferably the antibodies, the antibody fragments and T cell receptors bind the HLA/peptide complexes in a peptide-specific manner. Such antibodies are useful, for example, in identifying cells presenting the HLA/peptide complexes, particularly complexes comprising an HLA-A2, A3, A26, HLA-B7, B8, B15, B27, B35, B44 or B51 molecule, preferably HLA-A3, B35 or B51, and a peptide selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100), preferably a peptide selected from the group consisting of SEQ ID NO: 1 (p94-102), SEQ ID NO: 2 (p93-101), SEQ ID NO: 3 (p108-116) and SEQ ID NO: 4 (p91-99). Such antibodies are also useful in promoting the regression or inhibiting the progression of a tumor which expresses complexes of the HLA and peptide. Polyclonal antisera and monoclonal antibodies specific to the peptides or HLA/peptide complexes of this invention may be generated according to standard procedures. See e.g., Catty, D., Antibodies, A Practical Approach, Vol. 1, IRL Press, Washington D.C. (1988); Klein, J. Immunology: The Science of Cell-Non-Cell Discrimination, John Wiley and Sons, New York (1982); Kennett, R., et al., Monoclonal Antibodies, Hybridoma, A New Dimension In Biological Analyses, Plenum Press, New York (1980); Campbell, A., Monoclonal Antibody Technology, in Laboratory Techniques and Biochemistry and Molecular Biology, Vol. 13 (Burdon, R. et al. EDS.), Elsevier Amsterdam (1984); Eisen, H. N., Microbiology, third edition, Davis, B. D. et al. EDS. (Harper & Rowe, Philadelphia (1980); Kohler and Milstein, Nature, 256:495 (1975) all incorporated herein by reference.) Methods for identifying Fab molecules endowed with the antigen-specific, HLA-restricted specificity of T cells has been described by Denkberg et al. PNAS 99:9421-9426 (2002) and Cohen et al. Cancer Research 62:5835-5844 (2002) both incorporated herein by reference). Methods for generating and identifying other antibody molecules, e.g., scFv and diabodies are well known in the art, see e.g., Bird et al., Science, 242:423-426 (1988); Huston et al., Proc. Natl. Acad. Sci., 85:5879-5883 (1988); Mallender and Voss, J. Biol. Chem. 269:199-206 (1994); Ito and Kurosawa, J Biol Chem 27: 20668-20675 (1993), and; Gandecha et al., Prot Express Purif. 5: 385-390 (1994).

The antibodies of this invention can be used for experimental purposes (e.g. localization of the HLA/peptide complexes, immunoprecipitations, Western blots, flow cytometry, ELISA etc.) as well as diagnostic or therapeutic purposes, e.g., assaying extracts of tissue biopsies for the presence of HLA/peptide complexes, targeting delivery of cytotoxic or cytostatic substances to cells expressing the appropriate HLA/peptide complex. The antibodies of this invention are useful for the study and analysis of antigen presentation on tumor cells and can be used to assay for changes in the HLA/peptide complex expression before, during or after a treatment protocol, e.g., vaccination with peptides, antigen presenting cells, HLA/peptide tetramers, adoptive transfer or chemotherapy. The antibodies and antibody fragments of this invention may be coupled to diagnostic labeling agents for imaging of cells and tissues that express the HLA/peptide complexes or may be coupled to therapeutically useful agents by using standard methods well-known in the art. The antibodies also may be coupled to labeling agents for imaging e.g., radiolabels or fluorescent labels, or may be coupled to, e.g., biotin or antitumor agents, e.g., radioiodinated compounds, toxins such as ricin, methotrexate, cytostatic or cytolytic drugs, etc. Examples of diagnostic agents suitable for conjugating to the antibodies of this invention include e.g., barium sulfate, diatrizoate sodium, diatrizoate meglumine, iocetamic acid, iopanoic acid, ipodate calcium, metrizamide, tyropanoate sodium and radiodiagnostics including positron emitters such as fluorine-18 and carbon-11, gamma emitters such as iodine-123, technitium-99 m, iodine-131 and indium-111, nuclides for nuclear magnetic resonance such as fluorine and gadolinium. As used herein, “therapeutically useful agents” include any therapeutic molecule which are preferably targeted selectively to a cell expressing the HLA/peptide complexes, including antineoplastic agents, radioiodinated compounds, toxins, other cytostatic or cytolytic drugs. Antineoplastic therapeutics are well known and include: aminoglutethimide, azathioprine, bleomycin sulfate, busulfan, carmustine, chlorambucil, cisplatin, cyclophosphamide, cyclosporine, cytarabidine, dacarbazine, dactinomycin, daunorubicin, doxorubicin, taxol, etoposide, fluorouracil, interferon.alpha., lomustine, mercaptopurine, methotrexate, mitotane, procarbazine HCl, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60. The antibodies may be administered to a subject having a pathological condition characterized by the presentation of the HLA/peptide complexes of this invention, e.g., melanoma and serveral other cancers, as described in Jungbluth et al., Int. J. Cancer, 92:856-860 (Jun. 15, 2001, incorporated herein by reference), in an amount sufficient to alleviate the symptoms associated with the pathological condition.

Soluble T cell receptors (TcR) which specifically bind to the HLA/peptide complexes described herein are also an aspect of this invention. In their soluble form T cell receptors are analogous to a monoclonal antibody in that they bind to HLA/peptide complex in a peptide-specific manner. Immobilized TcRs or antibodies may be used to identify and purify unknown peptide/HLA complexes which may be involved in cellular abnormalities. Methods for identifying and isolating soluble TcRs are known in the art, see for example WO 99/60119, WO 99/60120 (both incorporated herein by reference) which describe synthetic multivalent T cell receptor complex for binding to peptide-MHC complexes. Recombinant, refolded soluble T cell receptors are specifically described. Such receptors may be used for delivering therapeutic agents or detecting specific peptide-MHC complexes expressed by tumor cells. WO 02/088740 (incorporated by reference) describes a method for identifying a substance that binds to a peptide-MHC complex. A peptide-MHC complex is formed between a predetermined MHC and peptide known to bind to such predetermined MHC. The complex is then use to screen or select an entity that binds to the peptide-MHC complex such as a T cell receptor. The method could also be applied to the selection of monoclonal antibodies that bind to the predetermined peptide-MHC complex.

Also an embodiment of this invention are nucleic acid molecules encoding the antibodies and T cell receptors of this invention and host cells, e.g., human T cells, transformed with a nucleic acid molecule encoding a recombinant antibody or antibody fragment, e.g., scFv or Fab, or a TcR specific for a predesignated HLA/peptide complex as described herein, particularly a complex wherein the HLA molecule is an HLA-A2, A3, A26, HLA-B7, B8, B15, B27, B35, B44 or B51 molecule, preferably HLA-A3, B35 or B51, and the peptide has a nucleotide sequence set forth in SEQ ID NO. 1, 2, 3, 4, 5, 6, 7 or 8, particularly SEQ ID NO: 1 or 2. Recombinant Fab or TcR specific for a predesignated HLA/peptide complex in T cells have been described in, e.g., Willemsen et al., “A phage display selected fab fragment with MHC class I-restricted specificity for MAGE-A1 allows for retargeting of primary human T lymphocytes” Gene Ther. November 2001; 8(21):1601-8. PMID: 11894998 [PubMed—indexed for MEDLINE] and Willemsen et al., “Grafting primary human T lymphocytes with cancer-specific chimeric single chain and two chain TCR”. Gene Ther. August 2000; 7(16):1369-77. PMID: 10981663 [PubMed—indexed for MEDLINE] (both incorporated herein by reference) and have applications in an autologous T cell transfer setting. The autologous T cells, transduced to express recombinant antibody or TcR, may be infused into a patient having an pathological condition associated with cells expressing the HLA/peptide complex. The transduced T cells are administered in an amount sufficient to inhibit the progression or alleviate at least some of the symptoms associated with the pathological condition.

An embodiment of this invention is a method for promoting regression or inhibiting progression of a tumor in a subject in need thereof wherein the tumor expresses a complex of HLA and peptide. The method comprises administering an an antibody, antibody fragment or soluble T cell receptor, which specifically binds to the HLA/peptide complex, or by administering cells transduced so that they express those antibodies or TcR in amounts that are sufficient to promote the regression or inhibit progression of the tumor expressing the HLA/peptide complex, e.g., a melanoma or other cancer, as described in Jungbluth et al., Int. J. Cancer 92(6)856-860 (Jun. 15, 2001). Preferably the HLA is an HLA-A3, B35 or B51 and the peptide is a NY-ESO-1 derived peptide preferably a peptide consisting of the sequences set forth in SEQ ID NO: 1, 2, 3, 4, 5, 6, 7, or 8, or their analogs, and particularly NY-ESO-1 p94-102 (SEQ ID NO: 1). The antibodies, antibody fragments and soluble T cell receptors may be conjugated with, or administered in conjunction with, an antineoplastic agent, e.g., radioiodinated compounds, toxins such as ricin, methotrexate, or a cytostatic or cytolytic agent as discussed supra. See e.g., Patan et al., Biochem. Biophys. Acta, 133:C1-C6 (1997), Lode et al., Innunol. Res. 21:279-288 (2000) and Wihoff et al. Curr. Opin. Mo. Ther. 3:53-62 (2001) (all incorporated herein by reference) for a discussion of the construction of recombinant immunotoxins, antibody fusions with cytokine molecules and bispecific antibody therapy or immunogene therapy.

CTLs per se that are specific for a complex of an HLA, particularly HLA-A3, HLA-B35 or HLA-B51, and a peptide having an amino acid sequence as set forth in SEQ ID NO: 1, 2 3 or 4 are also an aspect of this invention. The CTLs are useful in adoptive transfer wherein the CTLs are administered to a subject in need thereof in an amount that is sufficient for the CTLs to recognize cells presenting the complex and lysing the cells. The CTLs are also useful for identifying cells that present complexes of e.g. HLA-A2, A3, A26, B7, B8, B15, B27, B35, B44 or B51, particularly A3 or B51, and the peptides or peptide analogs of this invention.

This invention further relates to a method for treating a subject with a disorder characterized by the presence of complexes of an HLA molecule and a peptide selected from the group consisting of SEQ ID NO: 1 (p94-102), SEQ ID NO: 2 (p93-101), SEQ ID NO: 3 (p108-116), and SEQ ID NO: 4 (p91-99) on cell surfaces by administering to the subject an amount of cytolytic T cells, which are specific for the complexes of the HLA molecule, particularly HLA-A3, HLA-B35 and HLA-B51, and the peptide, wherein the amount is sufficient to alleviate the disorder. In another embodiment, the peptides, peptide analogs or polytopes may be administered to the subject in an amount that is sufficient to induce an immune response such as the production of antibodies or the stimulation of CD8+ T cells specific for cells expressing the HLA/peptide complex and alleviate the symptoms of the disorder.

This invention also relates to a method for treating a subject with a disorder characterized by the presence of complexes of on HLA molecule, e.g. HLA-A2, A3, A26, B7, B8, B15, B27, B35, B44 or B51, particularly an HLA-A3, HLA-B35 or HLA-B51 molecules and a peptide having an amino acid sequence consisting of an amino acid sequence selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100) presented on cell surfaces. The method comprises administering to the subject an amount of cytolytic T cells, which are specific for complexes of the HLA molecules and the peptide, wherein the amount is sufficient to alleviate the disorder. In another embodiment, the peptides, peptide analogs or polytopes may be administered to the subject in an amount that is sufficient to stimulate CD8+ T cells and alleviate the symptoms of the disorder.

This invention also relates to methods for inducing an immune response in a subject in need thereof, e.g. one having a disorder characterized by the presence of complexes of an HLA molecule, particularly an HLA-A3, HLA-B35 and/or HLA-B51 molecule, and a peptide of this invention, particularly a peptide having an amino acid sequence selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100). presented on cell surfaces. In one embodiment of this invention the method comprises administering a composition comprising an effective amount NY-ESO-1 to the subject wherein the amount is sufficient to induce an immune response, e.g., antibody production, or stimulation of CD8+ T lymphocytes specific for complexes of an HLA and a peptide of this invention, and alleviate the disorder. The method is particularly useful for a subject who has cancer cells that express NY-ESO-1 and particularly a subject whose cells express HLA-A3, HLA-B35 and/or HLA-B51 as well. The generation and proliferation of the CTLs and the production of antibodies may be monitored by any means known in the art.

A further embodiment of this invetion is a method for inducing an immune response in a subject in need thereof, e.g., a subject having a disorder characterized by the presence of complexes of an HLA molecule, particularly an HLA-A3, HLA-B35 and/or HLA-B51 molecule, and a peptide of this invention, particularly a peptide having an amino acid sequence selected from the group consisting of SEQ ID NO: 1 (p94-102) , SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100). presented on cell surfaces by administering a composition comprising the nucleic acid molecules of this invention wherein the nucleic acid molecule encodes NY-ESO-1 and/or a peptide or polytope of this invention. The nucleic acid molecule may be in the form of a vector, e.g. a plasmid, cosmid or recombinant viral vector. Methods for the construction of vectors suitable for the expression of a desired protien or peptide in a particular host cell are well known in the art. The nucleic acid molecule is preferably administered in an amount sufficient to induce an immune response, e.g., antibody production or stimulation of CD8+ T lymphocytes specific for complexes of the HLA and peptide. Preferably the immune response is sufficient to alleviate at least some symptoms of the disorder. The method is particularly useful for a subject who has cancer cells that express NY-ESO-1 and particularly a subject whose cells express HLA-A3, HLA-B35 and/or HLA-B51 as well. The generation and proliferation of the CTLs and production of antibodies may be monitored by any means known in the art.

Therapeutically or pharmaceutically effective amount as it is applied to the peptides, peptide analogs, tetramer complexes, compositions, nucleic acid molecules and CTLs of this invention refers to the amount of the peptides, peptide analogs, tetramer complexes, compositions, nucleic acid molecules and CTLs of this invention that is sufficient to induce a desired biological result. The biological result may be the alleviation of the signs, symptoms or causes of a disease, or any other desired alteration of a biological system. In the present invention that amount may be sufficient to induce an immune response such as the production of antibodies specific for NY-ESO-1 or the peptides or peptide analogs of this invention or a response by CTL cells, e.g., their proliferation or their lysis of target cells expressing an appropriate HLA/peptide complex, or sufficient to alleviate the symptoms of a disorder characterized by the expression of a complex of HLA, particularly HLA-A3, HLA-B35 or HLA-B51, and a peptide of this invention, e.g., a peptide consisting of the amino acid sequence set forth in SEQ ID NO: 1, 2, 3 or 4.

### EXAMPLES

### Example 1

A. NY-ESO-1 Serum Antibody

Serum antibody responses against the recombinant NY-ESO-1 protein were tested by standard Western blot analysis and ELISA, using NY-ESO-1 recombinant protein purified from E. coli as described (Stockert et al., J Exp Med 187(8): 1349-54, (1998) incorporated herein by reference).

B. Patients

Five patients with advanced NY-ESO-1 expressing cancer and detectable NY-ESO-1 serum antibody were selected for the assessment of peptide-specific CD8+ T cell responses. HLA class I types and diagnoses were: NW1539 (A*03, A*11; B*44, B*51; Cw*04, Cw*05) melanoma; NW1352 (A*03, A*31; B*15, B*35; Cw*03, Cw*04) melanoma; NW923 (A*01, A*03; B*51, B*52; Cw06, Cw012) urothelial carcinoma; NW1354 (A*03, A*11; B*35, B*44; Cw04, Cw016) non-small cell lung cancer; NW1274 (A*2; B*44, B*51; Cw1-7 neg) melanoma. PHA blasts as antigen presenting cells were prepared from peripheral blood lymphocytes (PBLs) of 2 healthy donors: NW1725 (A*32; B51:Cw1-7 neg and NW1726(A*2; B*7, Bw6; Cw7).

C. Cell Culture and Cell Lines

The tumor cell lines NW-MEL-38 and NW-MEL-1539 were cultured in Dulbecco's modified Eagle's medium (DMEM, GIBCO) containing 10 mM Hepes buffer, L-arginine (84 mg/l), L-glutamine (584 mg/l), penicillin (10 IU/ml), streptomycin (100 ug/ml), and 10% FCS. EBV-transformed B lymphocytes, MZ1257-EBV and NW115-EBV, used as feeder cells for the T cell culture, the mutant cell line CEMx721.174.T2 (T2) and CEMx721.174.T2 transfected with HLA-A3 (T2.A3), which was a generous gift from Dr. V. Cerundolo, were maintained in RPMI 1640 medium supplemented with 10 mM Hepes buffer, L-arginine (242 mg/l), L-asparagine (50 mg/l), L-glutamine (300 mg/l), penicillin (10 IU/ml), streptomycin (100 ug/ml), 1% non-essential amino acids, and 10% FCS.

The CD8+ T cell line NW1539-IVS-1 was generated from PBL of patient NW1539 by in vitro stimulation with the autologous tumor cell line NW-MEL-1539. The CD8+ T cell line NW1539-CTL-1/1 was obtained by limiting dilution and repetitive stimulation with the autologous tumor cell line MZ-MEL-19. The CD8+ T cell line NW1539-CTL-1/1 was obtained by limiting dilution and repetitive in vitro stimulation with NY-ESO-1 p94-102 (SEQ ID NO: 1).

D. Peptides

The production of peptides of 18 aminoacids in length with overlapping sequences, spanning the entire sequence of NY-ESO-1 have been described previously (Jager et al., J. Exp. Med. 191(4):625-30 (2000) incorporated herein by reference). NY-ESO-1 nonamer peptides of >95% purity were synthesized by Multiple Peptide Systems (San Diego). The sequences are presented in Table 1. The sequence of NY-ESO-1 is disclosed in U.S. Pat. No. 5,804,381 incorporated herein by reference.

TABLE 1

Peptides

| | |
|---|---|
| Position in NY-ESO-1 | Sequence |

| | | |
|---|---|---|
| 85-93 | SRLLEFYLA | (SEQ ID NO: 11) |
| 91-99 | YLAMPFATP | (SEQ ID NO: 4) |
| 92-100 | LAMPFATPM | (SEQ ID NO: 8) |
| 93-101 | AMPFATPME | (SEQ ID NO: 2) |
| 94-102 | MPFATPMEA | (SEQ ID NO: 1) |
| 97-106 | ATPMEAELAR | (SEQ ID NO: 12) |
| 108-116 | SLAQDAPPL | (SEQ ID NO: 3) |

E. Viral Vectors

Adenoviral constructs Ad2/EGFP (encoding green fluorescent protein) and Ad2/ESO (encoding NY-ESO-1), and vaccinia constructs vvWT (wild type vaccinia virus) and vv.ESO (encoding the full length NY-ESO-1 cDNA) have been described previously (Gnjatic et al., PNAS 97(20): 10917-22. (2000) incorporated herein by reference).

F. Presensitization with Peptides and Adenoviral Constructs

Purified CD8+ T lymphocytes were presensitized with peptide-pulsed or adenovirus-infected irradiated autologous PBL depleted of CD4+ and CD8+ T cells as described (Gnjatic et al., (2000) supra; Jager et al., PNAS 97(9): 4760-5 (2000) both incorporated herein by reference). Presensitized CD8+ T cells were used as effectors on day 6 for ELISPOT analysis, or restimulated on day 7 for the assessment of cytotoxicity against peptide pulsed T2.A3 cells (day 12), adenovirus-infected autologous APC or melanoma cells (day 13) in ⁵¹chromium release assays.

G. Target Cells

For the generation of monocyte-derived autologous antigen-presenting cells, peripheral blood mononuclear cells (PBMC), depleted of CD4+ and CD8+ T lymphocytes using magnetic beads (Minimacs, Miltenyi Biotec, Bergisch Gladbach, Germany), were seeded in 24-well plates at 4×10⁶ cells/well, and allowed to adhere to plastic for 24 hrs. Non-adherent cells were removed and remaining cells were used as antigen-presenting cells (APC), and cultured with GM-CSF 1000 U/ml (Leukomax, Sandoz, Nürnberg, Germany), and IL-4 1000 U/ml (Pharma Biotechnologie Hannover, Germany) for 5 days in X-vivo 15 medium (Bio Whittaker, Walkersville, Md., USA) 2 ml/well. For the assessment of HLA-A3 and B51 restricted presentation of NY-ESO-1, APC were treated on day 6 of in vitro culture with IL-4 1000 U/ml, IL-6 1000 U/ml, IL-1β10 ng/ml , TNFα10 ng/ml (IL-4, IL-6, IL-1β, TNFα obtained from Pharma Biotechnologie Hannover, Germany), GM-CSF 1000 U/ml, and prostaglandin 1 μg/ml (Sigma Chemical Co., St. Louis, Mo.). APC were infected with adenoviral constructs at 1000 infection units/cell, or pulsed with peptides at 10 μg/ml, and cultured for 24 hrs. APC were then washed twice and used as targets in ELIPOT assays at 3×10⁴ cells/well.

PHA blasts generated from PBL by incubation with 0.5 ug/ml PHA (Welcome) were pulsed with 10 μg/ml peptide. EBV-B cells were either pulsed with 10 μg/ml peptide or infected at 30 pfu/cell with adeno- or vaccinia wild-type or recombinant for NY-ESO-1, in 300 μl serum-free medium overnight.

H. ELISPOT Assay

Presensitized CD8+ T lymphocytes were added at 2.5×10⁴ cells/well, or T cell clones at 1000 cells/well, to 96-well flat-bottom nitrocellulose plates (MAHA S45 10, Millipore, Bedford Mass., USA) coated with 5 μg/ml of anti-interferon-gamma antibody (Hölzel Diagnostic, Köln, Germany) in a final volume of 100 μl NY-ESO-1 peptides were pulsed onto irradiated T2 and T2.A3 cells at a concentration of 10 μg/ml and added to the effector cells at 5×10⁴ cells/well. Plates were incubated for 16 h at 37° C., washed extensively (6 washes with a solution of 0.05% Tween 20/PBS), and supplemented with the biotinylated anti-interferon-gamma detection antibody (Hölzel Diagnostic, Köln, Germany) at 0.5 μg/ml. After incubation for 2 h at 37° C., plates were washed and developed with ABC-AP Vectastain (Vector, Burlingame, Calif., USA) for 1 h. After addition of substrate (BCIP/NPT) and 10 min of incubation, plates were prepared for microscopic counting of the blue spots. The number of blue spots/well was determined as the frequency of NY-ESO-1 specific CD8+ T cells/well, respectively.

I. Cytotoxicity Assay

Cytotoxicity against peptide pulsed T2.A3 cells, NY-ESO-1 transduced EBV-B cells, and tumor cells was determined in standard chromium release assays as described (Jager et al., PNAS 97(9): 4760-5 (2000)).

### Example 2

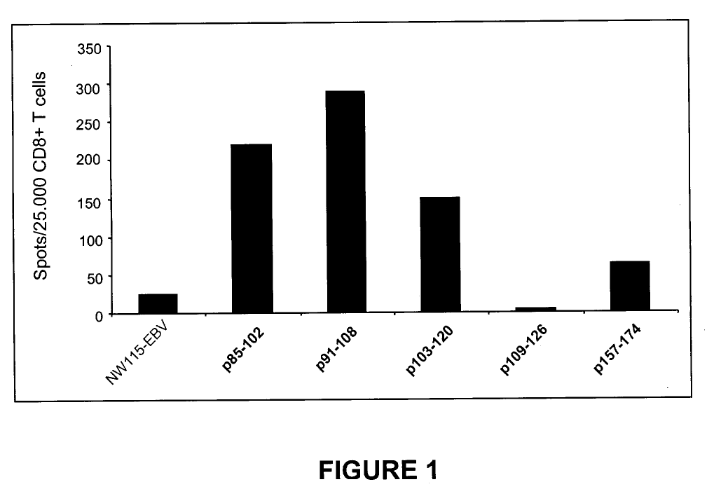

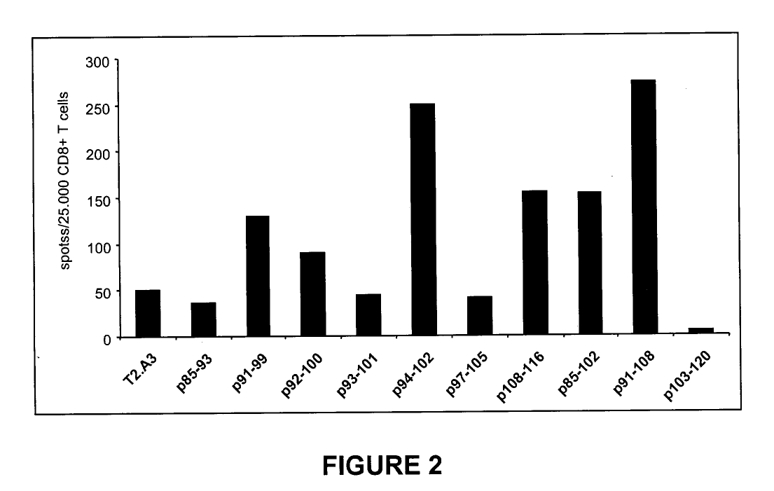

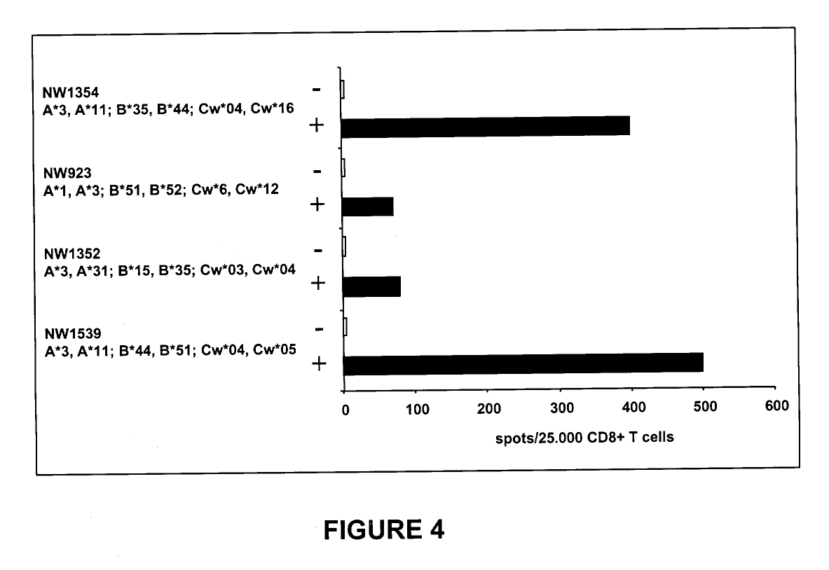

CD8+ selected effector T cells were sensitized with NY-ESO-1 recombinant adenovirus (Ad2/ESO)-infected CD8-depleted PBL of melanoma patient NW1352 (A3+, B51−) and tested for recognition of NY-ESO-1 epitopes on allogeneic HLA-A3+ NW115-EBV target cells pulsed with long overlapping 18-mer NY-ESO-1 peptides in ELISPOT assays. NY-ESO-1 p85-102 (SEQ ID NO: 5), p91-108 (SEQ ID NO: 6), and p103-120 (SEQ ID NO: 7) were recognized (FIG. 1). HLA-A3 was the only MHC class I allele shared by the target cell line and the T cells from patient NW1352, which indicated that the T cells recognize the peptide presented by A3. Furthermore, since these T cells were generated using adeno-ESO infected autologous stimulating cells expressing full length NY-ESO-1, one would expect that the stimulating peptide is naturally processed and presented on HLA-A3. Seven 9-mer peptides binding to HLA-A3 and located within the 18-mer sequences that were recognized by the T cells, were synthesized and tested for their recognition by Ad2/ESO-presensitized CD8+ NW1352 T cells. FIG. 2 shows the results of an ELISPOT assay against T2.A3 target cells pulsed with 7 NY-ESO-1 9-mer peptides and 3 long overlapping NY-ESO-1 peptides for control. Reactivity against NY-ESO-1 p94-102 (SEQ ID NO: 1) was confirmed by the analysis of Ad2/ESO presensitized CD8+ T cells of 4 patients sharing the HLA-A3 allele (FIG. 4).

### Example 3

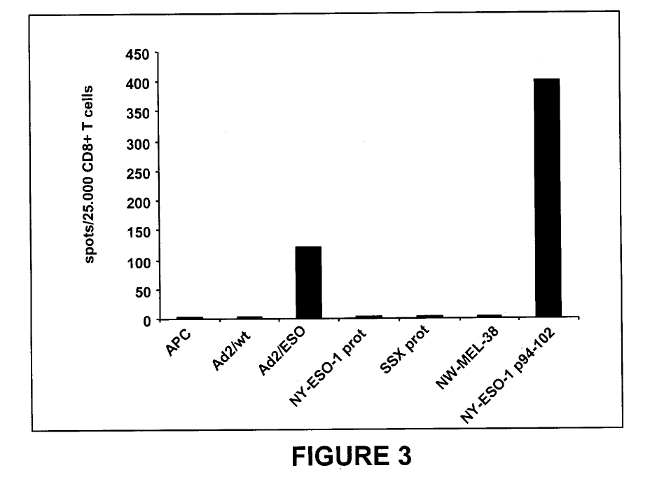

To assay the recognition of naturally processed NY-ESO-1 by peptide-sensitized CD8+ effector T cells, CD8+ T cell NW1539-CTL-1/15 was tested against NY-ESO-1 p94-102 (SEQ ID NO: 1), and tested on autologous monocyte-derived APC pulsed with the stimulating peptide or infected with Ad2/ESO in ELISPOT assays. FIG. 3 shows the specific recognition of NY-ESO-1 p94-102 (SEQ ID NO: 1) and Ad2/ESO-transfected APC, confirming that naturally processed NY-ESO-1 is recognized by peptide presensitized effector T cells. APC pulsed with the NY-ESO-1 recombinant protein, the SSX protein, or a lysate of the NY-ESO-1 expressing tumor cell line NW-MEL-38 were not recognized.

### Example 4

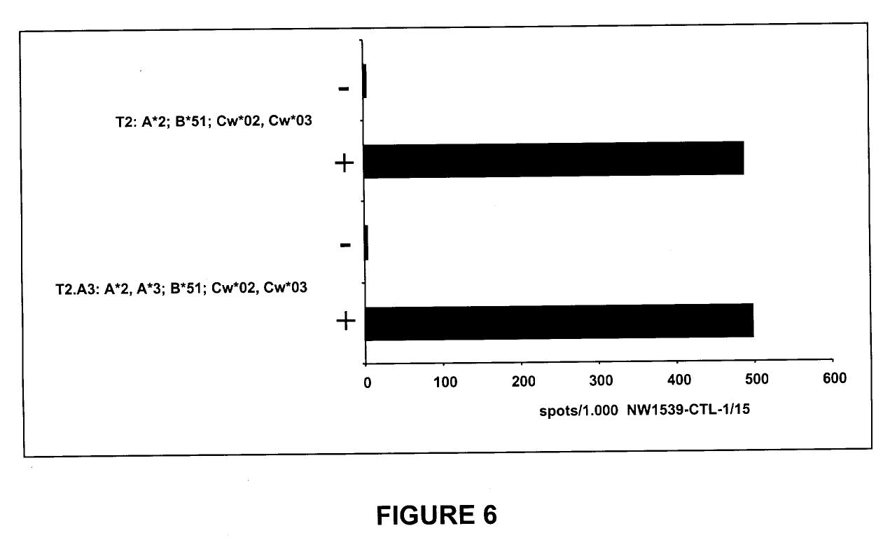

The specific recognition of NY-ESO-1 p94-102 (SEQ ID NO: 1) in the context of a second HLA allele, HLA-B51, was confirmed with the CD8+ T cell NW1539-CTL-1/15, tested against NY-ESO-1 p94-102 (SEQ ID NO: 1) peptide pulsed T2 and T2.A3 cells to confirm the restriction element. However, as shown in FIG. 6, both peptide pulsed T2 and T2.A3 cells were recognized with comparable efficacy. HLA class I analysis of target and effector cells showed HLA-B51 as the only shared allele, suggesting that NY-ESO-1 p94-102 is presented and recognized in the context of HLA-A3 and B51.

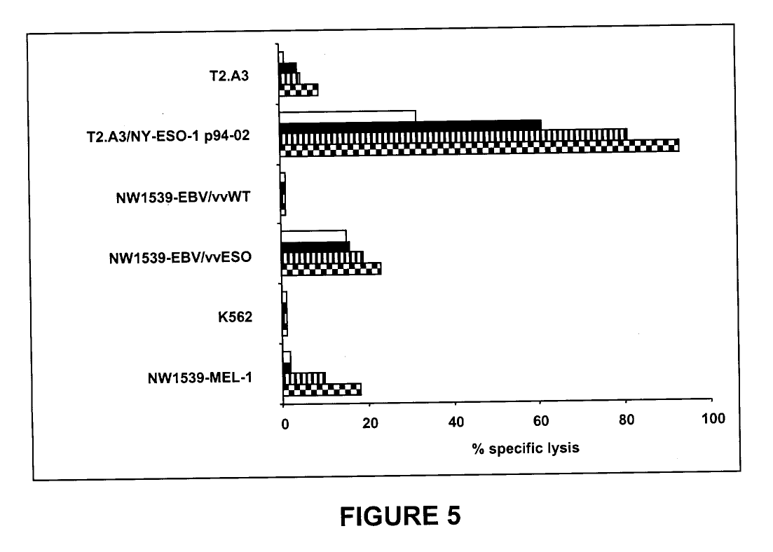

To confirm HLA-B51 as an alternative restriction element for NY-ESO-1 p94-102, peptide pulsed PHA blasts derived from different donors were assayed for recognition by peptide presensitized CD8+ T cells from melanoma patient NW1274. FIG. 5 shows a strong recognition of NY-ESO-1 p94-102 pulsed HLA-B51+ target cells NW1725 and NW1274. No CD8+ T cell reactivity was found against HLA-B51 negative target cells MZ7 and NW1726.

### Example 5

The following confirms that the restriction of naturally processed NY-ESO-1 is restricted to HLA-B51.

To define the HLA class I restriction element for NY-ESO-1 p94-102 (SEQ ID NO: 1) specific CD8+ T cell responses against naturally processed NY-ESO-1 COS-7 cells transfected with either HLA-A3 or B51 in combination with NY-ESO-1 were assayed. FIG. 5 shows that the HLA-A3+, B51+ effector T cell lines NW923 and NW1539 specifically recognize NY-ESO-1 in the context of HLA-B51, and not of HLA-A3. These results confirm that the NY-ESO-1 epitope p94-102 is naturally processed and presented by HLA-B51.

### Example 6

The specific cytotoxicity of T cell line NW1539-CTL-1/1 against NY-ESO-1 p94-102 and naturally processed NY-ESO-1 was assayed as follows. T cell line NW1539-CTL-1/15 was generated by limiting dilution and repetitive in vitro stimulation with NY-ESO-1 p94-102 (SEQ ID NO: 1). The cytotoxicity against NY-ESO-1 p94-102 (SEQ ID NO: 1) pulsed T2.A3 cells, autologous vvESO transduced NW1539-EBV-B cells and NW1539-MEL-1 melanoma cells was assayed and the results presented in FIG. 5. No lysis was observed against untreated T2.A3, K562, and NW1539-EBV-B cells.

### Example 7

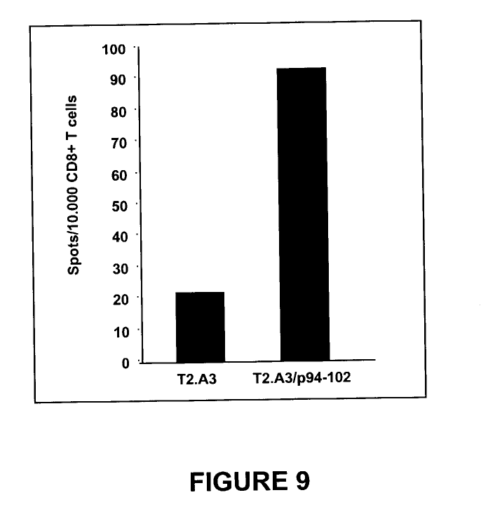

The spontaneous ex vivo reactivity of CD8+ NW1539 T cells against NY-ESO-1 p94-102 (SEQ ID NO: 1) was assayed as follows. Unsensitized CD8+ selected T cells from melanoma patient NW1539 were analyzed for specific recognition of NY-ESO-1 p94-102 in ELISPOT assays. The high number of peptide-specific spots shown in FIG. 9 suggests a precursor frequency of NY-ESO-1 p94-102-reactive T cells of approximately 0.7% of the CD8+ T cell population in this patient. Rare cases of comparable ex vivo precursor frequencies against Melan A/MART-1 p26-34 and NY-ESO-1 p157-167 were reported from single patients during prolonged vaccination with the respective peptides. In this light, the spontaneous CD8+ T cell reactivity against NY-ESO-1 p94-102 supports previous findings on the strong immunogenicity of NY-ESO-1 in cancer patients.

### Example 8

To further explore the role of the new NY-ESO-1 epitopes disclosed herein, SEQ ID NO: 1, 2, 3 and 4 for clinical vaccine trials, complementary assays (recombinant HLA-A3/peptide multimers, HLA-B51/peptide multimers, ELISPOT and cytotoxicity assays) are used to determine the immunogenicity of these peptide in larger series of NY-ESO-1 seropositive and −negative HLA-A3+ and/or HLA-B51 patients. The correlation of spontaneous NY-ESO-1 serum antibody with detectable CD8+ T cell reactivity in HLA-A3+ and HLA-B51+ cancer patients with the new NY-ESO-1 epitopes, demonstrates that the peptides consisting of an amino acid sequence set forth in SEQ ID NO: 1, 2, 3 and 4 are useful for the monitoring of spontaneous and vaccine-induced immune responses against NY-ESO-1 in HLA-A3+ and HLA-B51+ patients. This is similar to the results reported by Jager (Jager et al., PNAS 97(22): 12198-203 (2000))who assayed the immunological and clinical effects of intradermal immunization with the HLA-A2 restricted NY-ESO-1 epitopes p157-167 (SLLMWITQCFL, SEQ ID NO: 9), p157-165 (SLLMWITQC, SEQ ID NO: 10), and p155-163 (QLSLLMWIT, SEQ ID NO: 11) in a clinical trial. Jager et al. reported strong inflammatory delayed-type hypersensitivity reactions associated with primary CD8+ T cell responses against NY-ESO-1 peptides p157-167 SEQ ID NO: 9 and p157-165 SEQ ID NO: 10 were induced in the majority of patients without spontaneous immunity to NY-ESO-1. Stabilization and regression of metastatic disease observed in single patients coincided with and therefore was related to the induction of peptide specific CD8+ T cell responses in vivo.

The results presented herein demonstrate that Adeno-ESO stimulated NW1352 T cells (A3+, B51−), recognize NW115-EBV (A3+) targets pulsed with 18 mer NY-ESO-1 peptide(s) (FIG. 1). The T cells and target cells share only HLA-A3 indicating that the T cells recognize the peptide presented by A3. Furthermore, since these T cells were generated using adeno-ESO infected autologous stimulating cells one would expect that the peptide is naturally processed and presented on HLA-A3. The NW1352 T cells recognize T2-A3 targets pulsed with 9 mer NY-ESO-1 peptide p94-102 derived from the longer peptides (FIG. 2).

Adeno-ESO stimulated T cells from a second patient NW1539 (A3+, B51+) also recognized the p94-102 NY-ESO-1 peptide, either pulsed directly onto autologous APC and when naturally processed by adeno-ESO infected autologous APC targets (FIG. 6). Furthermore, peptide (p94-102) stimulated T cells from patients NW1352 and NW1539 and two additional patients NW1354 (A3+, B51−) and NW923 (A3+, B51+) recognized T2.A3 cells pulsed with p92-102 (FIG. 4).

Two of the A3+ patients NW1539 and NW923 from which p92-102 specific T cells were derived, also express HLA-B51. In the assays described supra, the T cells were derived using autologous stimulator cells either infected with adeno-ESO or pulsed with exogenous peptide, and then tested on either autologous or T2.A3 target cells, which also endogenously express HLA-B51. This suggested that the NW1539 and NW923 T cells recognized p92-102 presented by HLA-A3 and HLA-B51.

T cell NW1539CTL-1/15, derived from patient NW1539 (A3+, B51+), recognized autologous melanoma cell line NW1539, autologous EBV infected with vaccinia-ESO and T2.A3 pulsed with p94-102 confirming the specificity for p94-102 and the ability to recognize the naturally presented peptide (FIG. 5). These T cells also recognized T2 (B51+) and T2.A3 (A3+, B51+) when pulsed with p94-102 confirming that this peptide can be presented by both HLA-A3 and HLA-B51 (FIG. 6). FIG. 9 demonstrates that NW1539 (A3+, B51+) T cells derived ex-vivo recognized T2.A3 cells pulsed with p94-102.

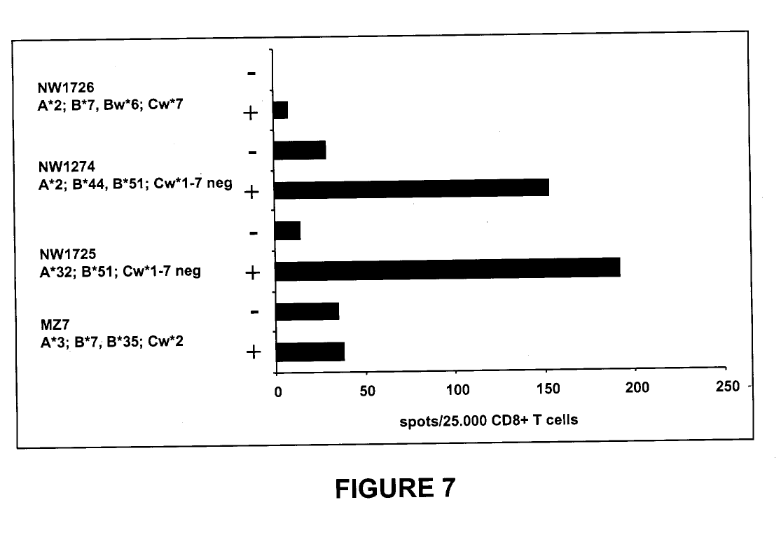

Presentation of p94-102 by HLA-B51 was confirmed using T cells derived from an additional patient NW1274 (A3−, B51+) by peptide (p94-102) stimulation. NW1274 CTL could recognize peptide (p94-102)-pulsed PHA blasts derived from patients NW1274 (A3−, B51+) and NW1725 (A3−, B51+), but not from patients NW1726 (A3−, B51−) or M27 (A3+, B51−) (FIG. 7).

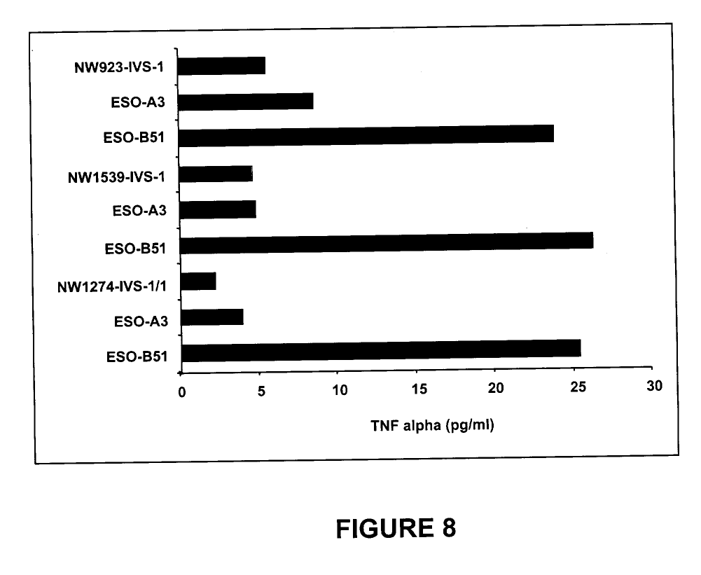

Recognition of naturally processed p94-102 presented on HLA-B51 was confirmed using T cells derived from patient NW1274 (A3−, B51+) by stimulation with an autologous tumor cell line. NW1274-IVS-1 T cells recognized COS-7 cells cotransfected with NY-ESO-1 and HLA-B51 cDNA, but not when cotransfected with NY-ESO-1 and HLA-A3 cDNA (FIG. 8). Similarly, T cells NW923-IVS-1 (A3+, B51+) and NW1539-IVS-1 (A3+, B51+), recognized COS-7 cells cotransfected with NY-ESO-1 and HLA-B51 cDNA, more effectively that COS-7 cotransfected with NY-ESO-1 and HLA-A3 cDNA. This last observation suggests that the p94-102/HLA-B51 epitope may dominate p94-102/HLA-A3 when both alleles are coexpressed in the same cell. Without wishing to be bound by theory, this is perhaps due to more efficient binding of p94-102 to HLA-B51 compared to HLA-A3. The p94-102 sequence MPFATPMEA (SEQ ID NO: 1) more closely fits the peptide-binding motif for HLA-B51 than HLA-A3 but it does not fit either motif particularly well.

Antigenic peptides derived from tumor antigens may be considered for vaccination of cancer patients if they represent naturally processed epitopes that can be recognized by CD8+ T cells on antigen expressing tumor cells (van der Bruggen et al., Science 254(5038): 1643-7.(1991); Wölfel et al., Int J Cancer 55(2): 237-44. (1993); Valmori et al., Cancer Res 60(16): 4499-506. (2000); Marchand et al., Int J Cancer 80(2): 219-30, (1999); Jager et al., Int J Cancer 86(4): 538-47, (2000); Coulie et al., J Exp Med 180(1): 35-42, (1994); Brichard et al., J Exp Med 178(2): 489-95, (1993); Bownds et al., J Immunother 24(1): 1-9, (2001)). Circumventing the difficulty of establishing tumor cell lines of many different tumor antigen- and HLA profiles, the gene of interest can be expressed in monocyte-derived antigen presenting cells or EBV-B cells and tested for recognition of naturally processed epitopes by HLA-matched CD8+ effector T cells (Gnjatic et al. (2000) supra). Two of the findings presented herein indicate that NY-ESO-1 p94-102 (SEQ ID NO: 1) represents a naturally processed epitope. These findings are (1) NY-ESO-1 p94-102 (SEQ ID NO: 1) peptide presensitized CD8+ T cells crossreact with NY-ESO-1 transduced autologous monocyte-derived APC, and (2) NW1539-IVS-1 T cells, which were exclusively stimulated with the autologous NY-ESO-1+ 1539-MEL-1 tumor cells, are reactive with NY-ESO-1 p94-102 (SEQ ID NO: 1) peptide pulsed target cells.

### Example 9

A. Peptides and Viral Vectors.

Synthetic NY-ESO-1 30-mer polypeptides p80-109 (ARGPESRLLEFYLAM PFATPMEAELARRSL, SEQ ID NO: 16), nonamer and decamer peptides included within p80-109, and peptide p157-165 (SLLMWITQC, SEQ ID NO: 10) were obtained from Bio-Synthesis (Lewisville, Tex.), with a purity of >90% as determined by mass spectrometry. Wild-type vaccinia virus (v.v.WT), and vaccinia virus recombinant for full-length NY-ESO-1 (v.v.ESO) were previously described (Gnjatic et al., Proc. Natl. Acad. Sci. USA 97:10917 (2000)).

B. In Vitro Sensitization of CD8+ T Cells.

CD8+ T lymphocytes were separated from peripheral blood lymphocytes (PBLs) of patient UC-98 by antibody-coated magnetic beads (Dynabeads; Dynal, Oslo, Norway) and seeded into round-bottomed 96-well plates (Corning, N.Y.) at a concentration of 5×10⁵ cells per well in RPMI medium 1640 supplemented with 10% human AB serum (NABI, Boca Raton, Fla.), L-glutamine (2 mM), penicillin (100 U/ml), streptomycin (100 μg/ml), and 1% nonessential amino acids. As antigen presenting cells (APCs), PBLs depleted of CD8+ T cells were pulsed with 10 μM peptide overnight at 37° C. in 250 μl serum-free medium. Pulsed APCs were then washed, irradiated and added to the plates containing CD8+ T cells, at a concentration of 1×10⁶ APCs per well. After 8 hours, IL-2 (10 U/ml) and IL-7 (20 ng/ml) were added to culture wells, and this step was repeated every three to four days, until the cells were harvested for testing.

C. Target Cells.

Target cells, EBV-transformed B lymphocytes and melanoma cell lines SK-MEL-106 and SK-MEL-139, were cultured in RPMI medium 1640 supplemented with 10% FCS, L-glutamine (2 mM), penicillin (100 U/ml), streptomycin (100 μg/ml), and 1% nonessential amino acids. HLA class I allele expression was determined by high-resolution DNA typing. For ELISPOT assays, the target cells were pulsed overnight with 10 μM peptide or infected with 30 pfu/cell v.v.WT or v.v.ESO, in 250 μl X-VIVO-15 (Bio-Whittaker).

D. ELISPOT Assays.

For ELISPOT assays, flat-bottomed, 96-well nitrocellulose plates were coated with IFN-γ mAb (2 μg/ml) and incubated overnight at 4° C. After washing with RPMI, plates were blocked with 10% human AB type serum for 2 h at 37° C. Presensitized CD8+ T cells (5×10⁴ and 1×10⁴) and 5×10⁴ targets cells (peptide-pulsed or v.v.ESO infected EBV-B, or tumor cells) were added to each well and incubated for 20 h in RPMI medium 1640 without serum. Plates were then washed thoroughly with water containing 0.05% Tween 20 to remove cells, and biotin labeled IFN-γ mAb (0.2 μg/ml) was added to each well. After incubation for 2 h at 37° C., plates were washed and developed with streptavidin-alkaline phosphatase (1 μg/ml; Mabtech) for 1 h at room temperature. After washing, substrate (5-bromo-4-chloro-3-indolyl phosphate/nitroblue tetrazolium; Sigma) was added and incubated for 5 min. After final washes, plate membranes displayed dark-violet spots that were counted under the microscope.

E. CD8+ T Cell Responses to NY-ESO-1 in Patient UC-98

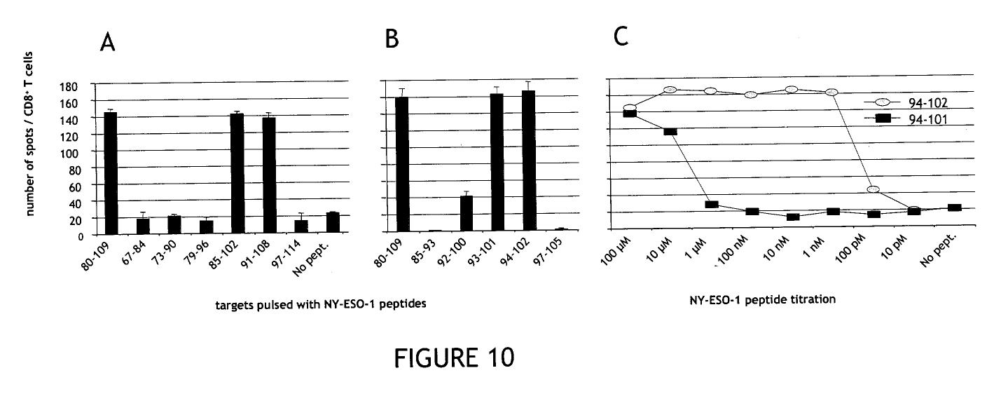

We previously established that there is a correlation between antibody and CD8+ T cell responses to NY-ESO-1 in cancer patients (Jäger et al., Proc. Natl. Acad. Sci. USA 97:4760 (2000) incorporated herein by reference). To determine NY-ESO-1 seropositive, HLA-A2 positive patient UC-98 would follow the same pattern, we presensitized CD8+ T cells as described above with NY-ESO-1 peptide p157-165, a known HLA-A2-restricted epitope, Jager et al., J. Exp. Med. 187(2):265-270 (Jan. 19, 1998). Surprisingly, no specific response could be detected to this peptide. To determine whether the CD8+ T cells of patient UC-98 responded to other NY-ESO-1 epitopes, we stimulated CD8+ T cells with a 30-mer peptide from NY-ESO-1, p80-109 (ARGPESRLLEFYLAM PFATPMEAELARRSL, SEQ ID NO: 16), which is processed by antigen presenting cells into multiple epitopes with various HLA restriction (Gnjatic et al., J. Immunol., 170:1191 (2003) incorporated herein by reference). A specific T cell response was observed in ELISPOT assays, indicating that an epitope within p80-109 of NY-ESO-1 was processed and recognized (FIG. 10A). To map this epitope, additional peptides with sequences spanning the central NY-ESO-1 region were synthesized and assayed for their recognition by CD8+ T cells (Table 2);

TABLE 2

| NY-ESO-1 | | |
|---|---|---|
| Position | Amino acid sequence | SEQ ID NO |
| 80-109 | ARGPESRLLEFYLAM | SEQ ID NO: 16 |
| | PFATPMEAELARRSL | |
| 157-165 | SLLMWITQC; | SEQ ID NO: 10 |
| 85-102 | SRLLEFYLAMPFATPMEA; | SEQ ID NO: 5 |
| 91-108 | YLAMPFATPMEAELARRS; | SEQ ID NO: 6 |
| 93-101 | AMPFATPME | SEQ ID NO: 2 |
| 93-102 | AMPFATPMEA | SEQ ID NO: 17 |
| 94-102 | MPFATPMEA | SEQ ID NO: 1 |

Two overlapping peptides, p85-102 (SEQ ID NO: 5) and p91-108 (SEQ ID NO: 6), were recognized, indicating that the epitope was within the amino acid sequence spanning positions 91-102 of NY-ESO-1 (FIG. 10A). Peptides p93-101 (SEQ ID NO: 2), p93-102(SEQ ID NO: 17) and p94-102 (SEQ ID NO: 1) were the minimal sequences detected by ELISPOT (FIG. 10B).

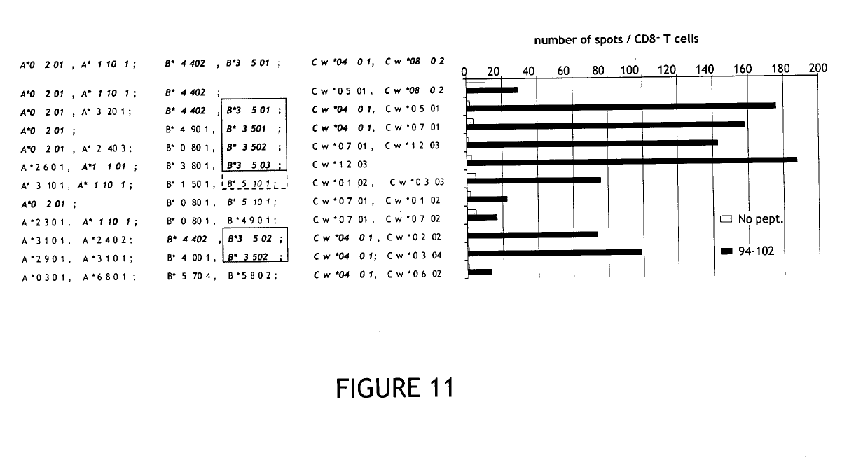

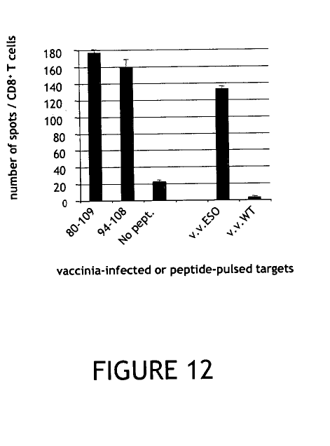

In titration assays, peptide p94-102 (SEQ ID NO: 1) was recognized by CD8+ T cells at concentrations as low as to 100 pM (FIG. 10C). Although this peptide was recently described as restricted by HLA-B51 (Jäger et al., Cancer Immunity 2:12 (2002). http://www.cancerimmunity.org/v2p12/020812.htm.), the cells of patient UC-98 did not express HLA-B51. Therefore we sought to define the HLA molecule in complex with p94-102 that was recognized by the patient's CD8+ T cells by testing partially histocompatible targets for their capacity to present p94-102 (FIG. 11). Only HLA-B35⁺ target cells were recognized when pulsed with p94-102, demonstrating a new HLA restriction for this peptide. Target cells expressing several HLA-B35 subtypes were recognized when pulsed with p94-102 indicating that the HLA restriction for this peptide is promiscuous to subtypes of HLA-B35, particularly the HLA-B*3501, 3502 and 3503 subtypes. Noteworthy, one HLA-B51⁺ target was also recognized, but to a lower extent, when pulsed with p94-102, suggesting a potential cross-reactivity between HLA-B35 and HLA-B51 for peptide presentation (FIG. 11). We confirmed that p94-102 was naturally processed from full-length NY-ESO-1 in antigen-presenting cells by demonstrating that CD8+ T cells specific for p94-102 recognized target cells infected with a recombinant vaccinia virus expressing NY-ESO-1 (FIG. 12).

The foregoing demonstrates that patient UC-98, who had serum antibodies for NY-ESO-1, had a concomitant CD8+ T cell response to NY-ESO-1. However, this response was directed against complexes of NY-ESO-1 p94-102 and HLA-B35, rather than against HLA-A2 and p157-165. It is not known whether the HLA-B35-restricted response is dominant over the HLA-A2 restricted response, or whether it is the only CD8+ T cell activity detectable in patient UC-98 at this sampling time point in the course of his disease.

Interestingly, as demonstrated supra, NY-ESO-1 p94-102 is also presented by HLA-B51 (Jäger et al., Cancer Immunity 2:12 (2002). http://www.cancerimmunity. org/v2p12/020812.htm.). HLA-B51 and HLA-B35 have very similar peptide binding motifs, and their cross-reaction was recently observed for an HIV-derived peptide (Ueno et al., J. Immunol. 169:4961 (2002) incorporated herein by reference). Similar super-type presentations were also observed for MAGE-A1 and MAGE-A3 peptides recognized by CD8+ clones in complex with an HLA-A1 or HLA-B35 molecule (Schultz et al., Tissue Antigens 57:103 (2001) and Luiten et al., Tissue Antigens 56:77 (2000) incorporated herein by reference.).

The immunogenicity of naturally processed NY-ESO-1 peptide p94-102 presented by HLA-A3, HLA-B51 and HLA-B35, which occur at a frequency of approximately 21%, 18% and 12% in some segments of the general population, offers great potential for peptide vaccination of a large proportion of cancer patients.

While the invention has been described with respect to certain embodiments, it should be appreciated that many modifications and changes may be made by those of ordinary skill in the art without departing from the spirit of the invention. It is intended that such modification, changes and equivalents fall within the scope of the claims presented herein. Other features of the invention will be clear to the skilled artisan and need not be reiterated here.

We claim:

1. An isolated peptide which consists of between eight and eleven amino acids, which binds to an HLA molecule selected from the group consisting of an HLA-A3, HLA-B35 or an HLA-B51 molecule and stimulates cytolytic T cells specific for complexes of said peptide and the HLA molecule, wherein at least eight contiguous amino acids of said peptide consist of at least eight contiguous amino acid sequence selected from the group consisting of SEQ ID NO: 1 (p94-102), SEQ ID NO: 2 (p93-101), SEQ ID NO: 3 (p108-116) and SEQ ID NO: 4 (p91-99).

2. The isolated peptide of claim 1, wherein the amino acid sequence of said peptide consists of an amino acid sequence selected from the group consisting of SEQ ID NO: 1 (p94-102), SEQ ID NO: 2 (p93-101), SEQ ID NO: 3 (p108-116) and SEQ ID NO: 4 (p91-99).

3. A composition comprising the isolated peptide of claim 1 and a carrier.

4. The composition of claim 3, further comprising an adjuvant.

5. The composition of claim 3, wherein the adjuvant is a cytokine.

6. The composition of claim 5, wherein said cytokine is granulocyte-macrophage colony-stimulating factor (GM-CSF) or interleukin-12 (IL-12).

7. The composition of claim 3, wherein the peptide has the amino acid sequence set forth in SEQ ID NO: 1 or SEQ ID NO: 2.

8. An isolated nucleic acid molecule which encodes the peptide of claim 1.

9. The isolated nucleic acid molecule of claim 8, wherein said peptide consists of the sequence set forth in SEQ ID NO: 1 (p94-102) or SEQ ID NO: 2 (p93-101).

10. An expression vector comprising the isolated nucleic acid molecule of claim 8, in operable linkage with a promoter.

11. The expression vector of claim 10, wherein said vector is a plasmid, a cosmid or a viral vector.

12. A host cell transformed or transfected with the nucleic acid molecule of claim 10.

13. A host cell transformed or transfected with the expression vector of claim 11.

14. An isolated cytolytic T cell specific for a complex of an HLA molecule selected from the group consisting of an HLA-A3, HLA-B35 and an HLA-B51 molecule and a peptide selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8.

15. The isolated cytolytic T cell of claim 14, wherein said peptide consists of the amino acid sequence set forth in SEQ ID NO: 1 or SEQ ID NO: 2.

16. A method for detecting a cytolytic T cell (CTL) specific for a complex of an HLA molecule selected from the group consisting of an HLA-A3, HLA-B35 and an HLA-B51 molecule, and an isolated peptide that consists of an amino acid sequence selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100) comprising,
(a) contacting a cytolytic T cell-containing sample with a composition comprising cells presenting a complex of the HLA molecule and said isolated peptide and
(b) determining if a CTL in said CTL containing sample recognizes said cells presenting the complex of the HLA molecule and the isolated peptide,
wherein recognition of said complex is indicative of a CTL specific for a complex of the HLA molecule and said isolated peptide.

17. The method of claim 16, wherein said peptide consists of the amino acid sequence set forth in SEQ ID NO: 1 or SEQ ID NO: 2.

18. The method of claim 16, wherein recognition of said complex is determined by assaying for a response selected from the group consisting of:
(a) CTL proliferation,
(b) CTL lysis of cells presenting a complex of the HLA molecule and said peptide, and
(c) release of cytokines by said CTLs.

19. The method of claim 18, wherein release of cytokines is assayed by ELISPOT.

20. The method of claim 16, wherein said CTL containing sample is from a subject having cancer.

21. The method of claim 16, wherein said CTL containing sample is from a subject who has been inoculated with a composition comprising said peptide.

22. The method of claim 16, wherein said cell presenting the complex of the HLA molecule and the peptide is transfected with a nucleic acid molecule which encodes said peptide.

23. The method of claim 16, wherein said cell presenting said complex is transfected with a nucleic acid molecule that encodes said peptide and transfected with a nucleic acid molecule that encodes the HLA molecule.

24. The method of claim 16, wherein steps (a)-(c) are repeated at predetermined intervals to monitor proliferation of said CTL specific for the complex of the HLA molecule and the peptide in said CTL containing sample.

25. A method for detecting a cytolytic T cell (CTL) specific for a complex of an HLA molecule selected from the group consisting of an HLA-A3, an HLA-B35 and an HLA-B51 molecule, and an isolated peptide that consists of an amino acid sequence selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100) comprising,
(a) contacting a cytolytic T cell-containing sample with a composition comprising tetramers of an HLA molecule, β2 microglobulin, biotin and the peptide,
(b) determining if a CTL in said CTL containing sample recognizes said tetramers,
wherein recognition of said tetramers is indicative of a CTL specific for a complex of the HLA molecule and said isolated peptide.

26. A polytope comprising at least two peptides that are linked together wherein at least one of said peptides is a peptide of claim 1.

27. The polytope of claim 26, wherein at least one of said peptide consists of the amino acid sequence set forth in SEQ ID NO: 1 or SEQ ID NO: 2.

28. The polytope of claim 26, comprising a peptide consisting of the amino acid sequence set forth in SEQ ID NO: 1 and a peptide consisting essentially the amino acid sequence set forth in SEQ ID NO: 2.

29. An isolated nucleic acid molecule that encodes the polytope of claim 26.

30. An expression vector comprising the nucleic acid molecule of claim 29 in operable linkage with a promoter.

31. The expression vector of claim 30 selected from the group consisting of a plasmid, a cosmid and a virus.

32. A host cell transformed or transfected with the isolated nucleic acid molecule of claim 30.

33. A host cell transformed or transfected with the expression vector of claim 31.

34. An isolated tetramer comprising an HLA molecule selected from the group consisting of an HLA-A3, an HLA-B35 and an HLA-B51 molecule, a peptide consisting of an amino acid sequence selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (p85-102), SEQ ID NO: 6 (p91-108), SEQ ID NO: 7 (p103-120) and SEQ ID NO: 8 (p92-100), biotin and a binding partner.

35. The isolated tetramer of claim 34 wherein the binding partner is avidin or strepavidin.

36. The tetramer of claim 34, wherein the peptide consists of the amino acid sequence set forth in SEQ ID NO: 1 or SEQ ID NO: 2.

37. A composition comprising the tetramer of claim 34 and a carrier.

38. A method for inducing an immune response in a subject in need thereof comprising administering a composition comprising an effective amount of a peptide consisting of an amino acid sequence selected from the group consisting of SEQ ID NO: 1 (p94-102), SEQ ID NO: 2 (p93-101), SEQ ID NO: 3 (p108-116) and SEQ ID NO: 4 (p91-99) and an adjuvant, wherein said effective amount is sufficient to induce an immune response in said subject.

39. The method of claim 38, wherein said peptide consists of the amino acid sequence set forth in SEQ ID NO: 1 or SEQ ID NO: 2.

40. The method of claim 38, wherein said immune response is a stimulation of CD8+ T lymphocytes specific for complexes of the HLA and said peptide.

41. The method of claim 38, wherein said immune response is production of an antibody specific for NY-ESO-1.

42. The method of claim 38, wherein said subject has cancer and wherein the subject's cancer cells express NY-ESO-1.

43. The method of claim 42, wherein said cancer cells expresses an HLA molecule selected from the group consisting of an HLA-A3, HLA-B35 and an HLA-B51.

44. The method of claim 38, wherein said composition comprises a non-proliferative cell presenting a complex of the peptide and an HLA molecule selected from the group consisting of HLA-A3 molecule, HLA-B35 molecule and HLA-B51 molecule.

45. The method of claim 38, wherein said non-proliferative cells are transfected with a nucleic acid molecule that encodes the peptide.

46. The method of claim 45, wherein said non-proliferative cells are transfected with a nucleic acid molecule that encodes the peptide and a nucleic acid molecule which encodes the HLA molecule.

47. The method of claim 46, wherein said non-proliferative cells are transfected with a nucleic acid molecule that encodes both the peptide and the HLA molecule.

48. The method of claim 38, wherein said composition comprises a complex of the peptide and an HLA molecule selected from the group consisting of an HLA-A3 molecule, an HLA-B35 molecule and an HLA-B51 molecule.

49. A method for treating a subject with a disorder characterized by the presence of complexes of an HLA molecule and a peptide having an amino acid sequence selected from the group consisting of SEQ ID NO: 1 (p94-102), SEQ ID NO: 2 (p93-101), SEQ ID NO: 3 (p108-116), SEQ ID NO: 4 (p91-99), SEQ ID NO: 5 (NY-ESO-1 p85-102), SEQ ID NO: 6 (NY-ESO-1 p91-108), SEQ ID NO: 7 (NY-ESO-1 p103-120), SEQ ID NO: 8 (NY-ESO-1 p92-100) and SEQ ID NO: 13, presented on cell surfaces, comprising administering to said subject an amount of a cytolytic T cell, which is specific for complexes of said HLA molecule and said peptide, wherein said amount is sufficient to alleviate said disorder.

50. The method of claim 49, wherein the HLA molecule is selected from the group consisting of an HLA-A3 molecule, an HLA-B35 molecule and an HLA-B51 molecule.

51. An isolated peptide which, when complexed to an MHC molecule, is recognized by a cytolytic T cell that recognizes a complex of an HLA-A3 molecule, an HLA-B35 molecule or an HLA-B51 molecule, and a peptide selected from the group consisting of SEQ ID NO: 1 (p94-102), SEQ ID NO: 2 (p93-101), SEQ ID NO: 3 (p108-116) and SEQ ID NO: 4 (p91-99).

52. A combinatorial library of derivatives of a peptide consisting of an amino acid sequence selected from the group consisting of SEQ ID NO: 1, 2, 3 or 4, wherein said derivatives consist of from eight to eleven amino acids.

53. The combinatorial library of claim 52 wherein said derivatives are chemically modified peptides.

54. The combinatorial library of claim 52 wherein said derivatives are presented on phage pIII or pVIII.

55. A screening assay for an analog of a peptide of claim 2, comprising
(a) contacting the combinatorial library of peptides of claim 52 with an HLA-A3 molecule, an HLA-B35 molecule and an HLA-B51 molecule,
(b) isolating members of the combinatorial library that bind to at least two of HLA-A3, HLA-B35 and HLA-B51 HLA molecule, and
(c) assaying the members that bind to both the HLA molecules for stimulation of CTL cells which are specific for complexes of HLA-A3, HLA-B35 or HLA-B51 and a peptide consisting of an amino acid sequence selected from the group consisting of SEQ ID NO: 1, 2, 3 or 4.
wherein stimulation of said CTLs is indicative of an analog of a peptide consisting of a sequence selected from the group consisting of SEQ ID NO: 1, 2, 3 or 4.

56. An isolated peptide which consists of between eight and eleven amino acids, which binds to an HLA molecule and stimulates cytolytic T cells specific for complexes of said peptide and the HLA molecule, wherein at least eight contiguous amino acids of said peptide consist of a sequence of at least eight contiguous amino acids of MPFSSPMEA (SEQ ID NO: 13).

57. The method of claim 56, wherein said HLA molecule is an HLA-A3, HLA-B35 or HLA-B51 molecule.

58. The method of claim 56, wherein the peptide consists of the sequence MPFSSPMEA (SEQ ID NO: 13).

59. An isolated peptide which, when complexed to an HLA molecule, is recognized by a cytolytic T cell that recognizes a complex of the HLA molecule and a peptide consisting of the sequence set forth in SEQ ID NO: 13.

60. The isolated peptide of claim 59, wherein said HLA molecule is HLA-A3, HLA-B35 or HLA-B51.

61. An isolated antibody or antibody fragment which specifically binds a HLA/peptide complex wherein said HLA is selected from the group consisting of HLA-A3, HLA-B35 or an HLA-B51 molecule and a peptide selected from the group consisting of SEQ ID NO: 1 (p94-102), SEQ ID NO: 2 (p93-101), SEQ ID NO: 3 (p108-116) and SEQ ID NO: 4 (p91-99).

62. The isolated antibody or antibody fragment of claim 61 wherein said antibody is a monoclonal antibody or an Fab.

63. An isolated antibody or antibody fragment which specifically binds to the isolated peptide of claim 1.

64. The isolated antibody or antibody fragment of claim 63 wherein said antibody is a monoclonal antibody or an Fab.

65. An isolated soluble T cell receptor which specifically binds to a HLA/peptide complex wherein said HLA is selected from the group consisting of HLA-A3, HLA-B35 or an HLA-B51 molecule and a peptide selected from the group consisting of SEQ ID NO: 1 (p94-102), SEQ ID NO: 2 (p93-101), SEQ ID NO: 3 (p108-116) and SEQ ID NO: 4 (p91-99).

66. A method for inducing an immune response in a subject having a disorder characterized by the presence of complexes of an HLA molecule and a peptide selected from the group consisting of SEQ ID NO: 1 (p94-102), SEQ ID NO: 2 (p93-101), SEQ ID NO: 3 (p108-116), SEQ ID NO: 4 (p91-99) and SEQ ID NO: 13 comprising administering a composition comprising an effective amount of the isolated nucleic acid molecule of claim 8, wherein said effective amount is sufficient to induce an immune response in said subject.

67. The method of claim 66 wherein the HLA is an HLA-A3, HLA-B35 or HLA-B51.

68. A method for inducing an immune response in a subject having a disorder characterized by the presence of complexes of an HLA molecule and a peptide selected from the group consisting of SEQ ID NO: 1 (p94-102), SEQ ID NO: 2 (p93-101), SEQ ID NO: 3 (p108-116), SEQ ID NO: 4 (p91-99) and SEQ ID NO: 13 on cell surfaces comprising administering a composition comprising an effective amount of the vector of claim 10 wherein said effective amount is sufficient to induce an immune response.

69. The method of claim 68 wherein said vector is a plasmid, a cosmid or a viral vector.

70. The method of claim 68 wherein the HLA is an HLA-A3, HLA-B35 or HLA-B51.

71. A method for inducing an immune response in a subject with a disorder characterized by the presence of complexes of an HLA molecule and a peptide selected from the group consisting of SEQ ID NO: 1 (p94-102), SEQ ID NO: 2 (p93-101), SEQ ID NO: 3 (p108-116), SEQ ID NO: 4 (p91-99) and SEQ ID NO: 13 on cell surfaces comprising administering a composition comprising an effective amount of NY-ESO-1 wherein said effective amount is sufficient to induce an immune response in said subject.

72. The method of claim 69 wherein said HLA is an HLA-A3, HLA-B35 or HLA-B51.


SEQUENCE LISTING (17 sequences)

SEQ ID NO: 1 - protein, 9 aa, Homo sapiens
MPFATPMEA

SEQ ID NO: 2 - protein, 9 aa, Homo sapiens
AMPFATPME

SEQ ID NO: 3 - protein, 9 aa, Homo sapiens
SLAQDAPPL

SEQ ID NO: 4 - protein, 9 aa, Homo sapiens
YLAMPFATP

SEQ ID NO: 5 - protein, 18 aa, Homo sapiens
SRLLEFYLAMPFATPMEA

SEQ ID NO: 6 - protein, 18 aa, Homo sapiens
YLAMPFATPMEAELARRS

SEQ ID NO: 7 - protein, 18 aa, Homo sapiens
ELARRSLAQDAPPLPVPG

SEQ ID NO: 8 - protein, 9 aa, Homo sapiens
LAMPFATPM

SEQ ID NO: 9 - protein, 11 aa, Homo sapiens
SLLMWITQCFL

SEQ ID NO: 10 - protein, 9 aa, Homo sapiens
SLLMWITQC

SEQ ID NO: 11 - protein, 9 aa, Homo sapiens
QLSLLMWIT

SEQ ID NO: 12 - protein, 10 aa, Homo sapiens
ATPMEAELAR

SEQ ID NO: 13 - protein, 9 aa, Homo sapiens
MPFSSPMEA

SEQ ID NO: 14 - protein, 180 aa, Homo sapiens
MQAEGRGTGGSTGDADGPGGPGEPDGPGGNAGGPGEAGATGGRAPRGAGAARASGPGGGAPRGPHGGAASGLNGCCRCGARGPESRLLEFYLAMPFATPMEAELARRSLAQDAPPLPVPGVLLKEFTVSGNELTERLTAADHRQLQLSESSCLQQLSLLMWETQCFLPVFLAQPPSGQRR

SEQ ID NO: 15 - DNA, 752 bp, Homo sapiens
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 16 - protein, 30 aa, Homo sapiens
ARGPESRLLEFYLAMPFATPMEAELARRSL

SEQ ID NO: 17 - protein, 10 aa, Homo sapiens
AMPFATPMEA